(19) United States
(12) Patent Application Publication
(10) Pub. No.: US 2026/0226553 A1
(43) Pub. Date: Aug. 6, 2026

(54) METHODS OF HYPER- AND HYPO-METHYLATION ANALYSIS FOR DISEASE DETECTION

(71) Applicants: THE REGENTS OF THE UNIVERSITY OF CALIFORNIA, Oakland, CA (US); EARLYDIAGNOSTICS, INC., Los Angeles, CA (US)

(72) Inventors: Xianghong Jasmine ZHOU, Encino, CA (US); Xiaohui NI, Los Angeles, CA (US); Chun-Chi LIU, Los Angeles, CA (US); Weihua ZENG, Los Angeles, CA (US); Mary Louisa STACKPOLE, Los Angeles, CA (US)

(73) Assignees: THE REGENTS OF THE UNIVERSITY OF CALIFORNIA, Oakland, CA (US); EARLYDIAGNOSTICS, INC., Los Angeles, CA (US)

(21) Appl. No.: 19/151,276

(22) PCT Filed: Jan. 26, 2024
(86) PCT No.: PCT/US2024/013147

§ 371 Date: Jul. 25, 2025

Related U.S. Application Data
(60) Provisional application No. 63/441,680, filed on Jan. 27, 2023.

(51) Int. Cl. C12Q 1/6886; C12Q 1/6806; C12Q 1/6855; G16B 40/20

(52) U.S. Cl. CPC C12Q 1/6886; C12Q 1/6806; C12Q 1/6855; G16B 40/20; C12Q 2600/118; C12Q 2600/154

(57) ABSTRACT

Aspects of the disclosure provide methods and compositions for hyper- and hypo-methylation analysis of nucleic acid molecules. In specific aspects, the nucleic acid molecules comprise cell-free DNA. The methods may comprise ligation of a first set of adapters, methylation-sensitive or methylation-dependent restriction enzyme digestion, ligation of a second set of adapters, amplification and sample partitioning, enrichment of nucleic acid molecules tagged with specific adapters, sequencing and analysis of the sequencing data, and detection of diseases such as cancer.

### CROSS-REFERENCE

This application claims the benefit of U.S. Provisional Application No. 63/441,680, filed Jan. 27, 2023, which is incorporated by reference herein in its entirety.

### STATEMENT REGARDING FEDERALLY SPONSORED RESEARCH OR DEVELOPMENT

This invention was made with govenrment support under CA230705 awarded by the Nationl Institutes of Health. The government has certain rights in the invention.

### BACKGROUND OF THE INVENTION

### I. Field of the Invention

Aspects of the disclosure include at least the fields of nucleic acid preparation and analysis, sequencing, molecular biology, cell biology, and medicine.

### II. Background

With the rapid development of next-generation sequencing (NGS) technologies, analysis of genomic alterations in DNA may be performed to provide diagnostic information about disease (e.g., cancer) or other physiological (e.g., fetal genetic materials in maternal blood) status. Some physiological conditions, including diseases or disorders such as cancers or infectious diseases, can cause release of DNA into the circulation (e.g., bloodstream or lymphatic system), where tumor DNA or microbiome DNA may become part of circulating cell-free DNA (cfDNA) in bodily fluids such as plasma or urine. Such cfDNA may be subjected to genomic or epigenomic profiling for clinical applications such as cancer screening, microbial detection, or prenatal testing. For example, the analysis of cfDNA hyper- or hypo-methylation status may be utilized in the early detection of cancer (Silva et al., British Journal of Cancer 80, 1262 (1999); Kang et al., Genome Biology 18:53 (2018); Li et al., Nucleic Acids Research 46:e89 (2018); Guo et al., Nature Genetics 49:635(2017 ); Liu et al., Annals of Oncology 31:745 (2020); Chen et al., Nature Communications 11:3475 (2020); Stackpole et al., Nature Communications 13:5566 (2022), each of which is incorporated by reference herein in its entirety). The DNA sample from a biological sample, such as blood, may comprise a mixture of DNAs from white blood cells (WBCs) and different tissues. The DNA of interest may be present in a heavy background of non-informative DNA. For example, cell-free DNA from cancer patients may contain only a minor fraction of tumor DNA, while most DNA may be from WBCs and various normal organs/tissues. In tissue biopsies, DNA from a pathologically diseased tissue sample may contain a heavy background of DNA from the healthy tissue. The disease-specific methylation information may be obscured by the background DNA methylation. To enrich hyper-/hypo-methylated nucleic acid molecules of interest, e.g., tumor-derived cell-free DNA, from hypo-/hyper-methylated background DNA in a DNA sample, the DNA sample can be divided into multiple aliquots and restriction enzymes may be used to eliminate background DNA. For example, methylation-sensitive restriction enzymes may be used to eliminate hypo-methylated background DNA for hyper-methylation analysis of DNA of interest (as described by, for example, U.S. Pat. No. 8,088,581B2; and International Publication No. WO 2022/073011 A1, each of which is incorporated herein by reference in its entirety). In some applications, such as cell-free DNA-based detection, only a limited amount of DNA may be available. Therefore, there remains a need for improved methods and compositions that are capable of obtaining hyper and hypo methylation information from an entire informative portion, rather than a sub-portion, of the DNA sample.

The present disclosure provides improved methods and compositions for hyper and hypo DNA methylation analysis for disease detection.

### SUMMARY OF THE INVENTION

Aspects of the present disclosure provide improvements on methods and compositions for hyper- and hypo-methylation analysis by enriching both hyper- and hypo-methylated nucleic acid molecules of interest from hypo- and hyper-methylated background DNA in a DNA mixture sample. In liquid biopsy applications with disease-specific (e.g. tumor-derived) cell-free DNA, the background DNA can be derived from cell-free DNAs from white blood cells (WBC) or healthy tissues. In tissue biopsy applications, for example, in diagnostics applications using DNAs from tissues of any kind, the background DNA can comprise DNAs from the surrounding healthy tissue, such as from an organ having both diseased and healthy tissues. Aspects of the present disclosure provide methods to eliminate such background DNAs or enrich DNA of interest based on their specific methylation patterns by using methylation-sensitive and/or methylation-dependent restriction enzymes. In certain aspects, the hypo-methylated background DNA are eliminated for hyper-methylation analysis in the methods of the disclosure. In certain aspects, the hyper-methylated background DNA are eliminated for hypo-methylation analysis in the methods of the disclosure. The hyper- and hypo-methylated DNA of interest can be used for downstream analysis, e.g., next-generation sequencing to detect disease-specific methylation. In particular aspects, the present disclosure provides methods of analyzing hyper- and hypo-methylation patterns of cell-free DNA (cfDNA) molecules, by eliminating background methylation signals from DNA of white blood cells or healthy tissues, for example, to provide information about cancer and other physiological states. In particular cases, the method is utilized for detecting cancer.

In an aspect, the present disclosure provides a method of enriching hyper- and hypo-methylated nucleic acid molecules in a plurality of nucleic acid molecules, comprising: (a) ligating the first set of adapters to the ends of said plurality of nucleic acid molecules; (b) digesting said plurality of nucleic acid molecules with one or more methylation-sensitive or methylation-dependent restriction enzymes to produce a plurality of nucleic acid molecules with digestion-enzyme-specific overhangs, and ligating the second set of adapters to said plurality of nucleic acid molecules with digestion-enzyme-specific overhangs; (c) amplifying the adapter-ligated plurality of nucleic acid molecules to produce the amplified adapter-ligated DNA fragments by utilizing one or more primers that bind to the first set of adapters and the second set of adapters; (d) partitioning the amplified adapter-ligated DNA fragments into at least two partitions; (e) enriching the amplified DNA fragments that have the first set of adapters on both ends in the first partition and enriching the amplified DNA fragments that have the second set of adapters on both ends in the second partition; and (f) processing the enriched DNA fragments to determine the methylation status of the enriched DNA fragments.

In an aspect, the present disclosure provides a method for enriching hyper-methylated and/or hypo-methylated nucleic acid molecules in a plurality of nucleic acid molecules, comprising: (a) ligating a first set of adapters to each end of each nucleic acid in the plurality of nucleic acid molecules to generate a first set of ligated molecules; (b) digesting the first set of ligated molecules with one or more methylation-sensitive restriction enzymes and/or methylation-dependent restriction enzymes to produce a plurality of nucleic acid molecules with digestion-enzyme-specific overhangs, and ligating a second set of adapters to said plurality of nucleic acid molecules with digestion-enzyme-specific overhangs to generate a second set of ligated molecules, wherein the second set of ligated molecules do not have a sequence that is recognized by the one or more methylation-sensitive restriction enzymes; (c) amplifying the first set of ligated molecules and the second set of ligated molecules to produce amplified adapter-ligated DNA fragments, at least in part using one or more primers that bind to the first set of adapters and one or more primers that bind to the second set of adapters; (d) partitioning the amplified adapter-ligated DNA fragments into at least two partitions; and (e) enriching the amplified adapter-ligated DNA fragments that have the first set of adapters on both ends in a first partition to generate enriched DNA fragments in the first partition.

In some embodiments, the method further comprises enriching hyper-methylated nucleic acid molecules, and (b) further comprises digesting the plurality of nucleic acid molecules with one or more methylation-sensitive restriction enzymes to produce a plurality of nucleic acid molecules with digestion-enzyme-specific overhangs. In some embodiments, (b) further comprises ligating the second set of adapters to the plurality of nucleic acid molecules with digestion-enzyme-specific overhangs to generate a second set of ligated molecules. In some embodiments, the second set of ligated molecules do not have a sequence that is recognized by the one or more methylation-sensitive restriction enzymes.

In some embodiments, the method further comprises enriching hypo-methylated nucleic acid molecules, and (b) further comprises digesting the plurality of nucleic acid molecules with one or more methylation-dependent restriction enzymes to produce a plurality of nucleic acid molecules with digestion-enzyme-specific overhangs. In some embodiments, (b) further comprises ligating the second set of adapters to the plurality of nucleic acid molecules with digestion-enzyme-specific overhangs to generate a second set of ligated molecules. In some embodiments, the second set of ligated molecules do not have a sequence that is recognized by the one or more methylation-sensitive restriction enzymes.

In some embodiments, the second set of adapters have overhangs that are complementary to overhangs generated by the methylation-sensitive restriction enzymes and/or methylation-dependent restriction enzymes, and (b) further comprises digesting, with one or more additional restriction enzymes, adapters from the first set of adapters and/or second set of adapters that have ligated together while not digesting a junction between a DNA fragment and an adapter.

In some embodiments, amplifying the first set of ligated molecules and the second set of ligated molecules to produce amplified adapter-ligated DNA fragments comprises 1, 2, 3, 4, 5, 6, 7, 8, 9, 10, 11, 12, 13, 14, 15, 16, 17, 18, 19, 20, or more (or any range derivable therein) amplification cycles. In some embodiments, amplifying the first set of ligated molecules and the second set of ligated molecules to produce amplified adapter-ligated DNA fragments comprises 1 to 3, 1 to 5, or 1 to 10 (or any range derivable therein) amplification cycles.

In some embodiments, (e) further comprises enriching the amplified DNA fragments that have the second set of adapters on both ends in a second partition to generate enriched DNA fragments in the second partition. Enriching the amplified adapter-ligated DNA fragments may comprise amplifying the first partition of amplified adapter-ligated DNA fragments utilizing primers that are capable of initiating polymerization at the first set of adapters. Enriching the amplified adapter-ligated DNA fragments may comprise amplifying the second partition of amplified adapter-ligated DNA fragments utilizing primers that are capable of initiating polymerization at the second set of adapters. Enriching the amplified adapter-ligated DNA fragments may comprise amplifying any partition of amplified adapter-ligated DNA fragments utilizing primers that are capable of initiating polymerization at the first and/or second set of adapters.

In some embodiments, (d) further comprises partitioning the amplified adapter-ligated DNA fragments into three or more partitions. In some embodiments, (e) further comprises enriching the amplified adapter-ligated DNA fragments in a third partition that have the first set of adapters in one end and have the second set of adapters in another end to generate enriched DNA fragments the third partition. In some embodiments, (e) further comprises, in a third partition, amplifying amplified adapter-ligated DNA fragments that have the first set of adapters in one end and have the second set of adapters in another end, using primers that are capable of initiating polymerization at the first set of adapters in one end and initiating polymerization at the second set of adapters in another end of the amplified adapter-ligated DNA fragments.

In some embodiments, (e) further comprises capturing at least a subset of the amplified adapter-ligated DNA fragments in one or more targeted genomic regions by purification. The purification may be done by immunoprecipitation and/or column chromatography. In some embodiments, the subset of amplified adapter-ligated DNA fragments are captured using hybrid capture probes. The hybrid capture probes may cover one or more restriction enzyme cutting sites in the targeted genomic regions.

In some embodiments, the method further comprises (f) processing the enriched DNA fragments to determine a methylation status of the enriched DNA fragments. The processing may comprise generating sequencing data that provides counts of the enriched DNA fragments. In some embodiments, (b) is performed using one or more methylation-sensitive restriction enzymes, and (f) further comprises determining the methylation status by counting hyper-methylated DNA fragments in a first partition and counting hypo-methylated DNA fragments in a second partition. In some embodiments, (b) is performed using one or more methylation-dependent restriction enzymes, and (f) further comprises determining the methylation status by counting hypo-methylated DNA fragments in a first partition and counting hyper-methylated DNA fragments in a second partition.

In some embodiments, the plurality of nucleic acid molecules are from a biological sample. The biological sample may be from an individual, such as a subject or patient. The biological sample may comprise blood. The biological sample may comprise cell-free DNA. The biological sample may comprise a biopsy. The biopsy may comprise a tumor biopsy. In some embodiments, the plurality of nucleic acid molecules comprises cell-free DNA. In some embodiments, the nucleic acid molecules are subject to fragmentation comprising fragmenting and shearing the nucleic acid molecules using different methods, for example, sonication with shearing devices and/or digestion with restriction enzymes. In some embodiments, the fragmentation fragments at least a part of the nucleic acid molecules to small sizes for further analysis.

In some embodiments, the first set of adapters is ligated to the ends of said plurality of nucleic acid molecules. Each of the adapters may comprise a functional sequence that is configured to couple to a flow cell of a nucleic acid sequencer. In some embodiments, the method further comprises, prior to adapter ligation, performing end repair or nucleic acid base tailing of said plurality of nucleic acid molecules. The adapter ligation comprises the ligation of sequencing adapters with any ligase, for example, T4 DNA.

In some embodiments, digesting the plurality of nucleic acid molecules with one or more methylation-sensitive restriction enzymes comprises performing digestion of at least a subset of the plurality of nucleic acid molecules with methylation-sensitive enzymes that are not able to cleave methylated cytosine residues. In some embodiments, the method comprises using one or more restriction enzymes of methylation sensitive restriction enzymes (MSRE) comprising one or more of AcII, HindIII, MluCI, PciI, AgeI, BspMI, BfuAI, SexAI, MluI, BceAI, HpyCH4IV, HpyCH4III, BaeI, BsaXI, AflIII, SpeI, BsrI, BmrI, BglII, BspDI, PI-SceI, NsiI, AseI, CspCI, MfeI, BssS^{α}I, DraIII, EcoP15I, AlwNI, BtsIMutI, NdeI, CviAII, FatI, NlaIII, FspEI, XcmI, BstXI, PflMI, BccI, NcoI, BseYI, FauI, TspMI, XmaI, LpnPI, AclI, ClaI, SacII, HpaII, MspI, ScrFI, StyD4I, BsaJI, BslI, BtgI, NciI, AvrII, MnlI, BbvCI, SbfI, Bpu10I, Bsu36I, EcoNI, HpyAV, BstNI, PspGI, StyI, BcgI, PvuI, EagI, RsrII, BsiEI, BsiWI, BsmBI, Hpy99I, AbaSI, MspJI, SgrAI, BfaI, BspCNI, Xhol, PaeR7I, EarI, AcuI, PstI, BpmI, DdeI, SfcI, AfIII, BpuEI, SmlI, Aval, BsoBI, MboII, BbsI, BsmI, EcoRI, Hgal, AatII, PfIFI, Tth111I, AhdI, DrdI, SacI, BseRI, PleI, HinfI, Sau3AI, MboI, DpnII, TfiI, BsrDI, BbvI, Bt^{α}I, BstAPI, SfaNI, SphI, NmeAIII, NgoMIV, BglI, AsiSI, BtgZI, HhaI, HinP1I, BssHII, NotI, Fnu4HI, Mwol, BmtI, NheI, BspQI, BlpI, TseI, ApeKI, Bsp1286I, AlwI, BamHI, BtsCI, FokI, FseI, SfiI, NarI, PluTI, KasI, AscI, EciI, BsmFI, ApaI, PspOMI, Sau96I, KpnI, Acc65I, BsaI, HphI, BstEII, AvaII, BanI, BaeGI, BsaHI, BanII, CviQI, BciVI, Sa1l, BcoDI, BsmAI, ApaLI, BsgI, AccI, Tsp45I, BsiHKAI, TspRI, ApoI, NspI, BsrF^{α}I, BstYI, HaeII, EcoO109I, PpuMI, I-CeuI, I-SceI, BspHI, BspEI, MmeI, Taq^{α}I, Hpy188I, Hpy188III, XbaI, BclI, PI-PspI, BsrGI, MseI, PacI, BstBI, PspXI, BsaWI, EaeI, HpyF30I, Sfr274I, or a functional analog thereof, or a combination thereof, or a mixture thereof. In some embodiments, digesting the plurality of nucleic acid molecules produces digestion-enzyme-specific 5′- or 3′-overhangs on the ends of the said plurality of nucleic acid molecules. For example, the HpaII digestion may produce a 5′-CG overhangs.

In some embodiments, digesting the plurality of nucleic acid molecules with one or more methylation-dependent restriction enzymes comprises performing digestion of at least a subset of the plurality of nucleic acid molecules with methylation-dependent enzymes that are only able to cleave methylated cytosine residues. In some embodiments, the method comprise using one or more restriction enzymes of methylation dependent restriction enzymes (MSRE) comprising one or more of LpnPI, McrBC, GlaI, PkrI, MteI, AoxI, or a functional analog thereof, or a combination thereof, or a mixture thereof. In some embodiments, digesting the plurality of nucleic acid molecules produces digestion-enzyme-specific 5′- or 3′-overhangs on the ends of the said plurality of nucleic acid molecules.

In some embodiments, digesting the plurality of nucleic acid molecules and ligating the second set of adapters occur in the same reaction, and the second set of adapters have overhangs that complement the digestion-enzyme-specific 5′- or 3′-overhangs on the ends of the said plurality of nucleic acid molecules, and (b) further comprises digesting the junction between the end of one adapter and the end of another adapter but does not digest the junction between the end of the DNA fragment and the end of the adapter with one or more additional restriction enzymes. The one or more additional restriction enzymes may comprise one or more of BspDI, ClaI, AclI, NarI, XhoI, SmlI, HpyF30I, PaeR7I, Sfr274I, or a functional analog thereof or a mixture thereof. Each of the second set of adapters may comprise a functional sequence that is configured to couple to a flow cell of a nucleic acid sequencer and is distinguishable by sequencer from that of the first set of adapters.

In some embodiments, digesting the plurality of nucleic acid molecules and ligating the second set of adapters occur in different reactions. In some embodiments, digesting the plurality of nucleic acid molecules and ligating the second set of adapters occur in different reaction vessels.

In some embodiments, after digesting the plurality of nucleic acid molecules and ligating the second set of adapters, and before amplifying the adapter-ligated plurality of nucleic acid molecules, the method further comprises subjecting the plurality of nucleic acid molecules to conditions sufficient to permit the methylated nucleic acid bases to be distinguishable from the unmethylated nucleic acid bases. In some embodiments, subjecting the nucleic acid molecules to conditions sufficient to distinguish methylated vs. unmethylated bases comprises performing bisulfite conversion on the nucleic acid molecules. In some embodiments, subjecting the nucleic acid molecules to conditions sufficient to distinguish methylated vs. unmethylated bases comprises enzymatic and/or chemical reactions to oxidize the methylated cytosine nucleic acid bases and/or hydroxymethylated cytosine nucleic acid bases followed by reduction and/or deamination of oxidation reaction products.

In some embodiments, amplifying the adapter-ligated plurality of nucleic acid molecules comprises amplification, such as PCR. In some embodiments, the primers for PCR are designed to recognize both the first set of adapters and the second set of adapters, and limited PCR cycles are performed to amplify the plurality of nucleic acid molecules to a sufficient amount for the partitioning step. Examples of limited PCR cycles may include about 1 cycle, about 2 cycles, about 3 cycles, about 4 cycles, and about 5 cycles.

In some embodiments, enriching the amplified DNA fragments that have the first set of adapters on both ends comprises amplifying the first partition of amplified DNA fragments utilizing primers that are capable of initiating polymerization at the first set of adapters, and enriching the amplified DNA fragments that have the second set of adapters on both ends comprises amplifying the second partition of amplified DNA fragments utilizing primers that are capable of initiating polymerization at the second set of adapters.

In some embodiments, (d) further comprises partitioning the amplified adapter-ligated DNA fragments into three or more partitions, and (e) further comprises enriching the amplified DNA fragments in the third partition that have the first set of adapters in one end and have the second set of adapters in another end in the third partition. In some embodiments, enriching the amplified DNA fragments in the third partition that have the first set of adapters in one end and have the second set of adapters in another end comprises amplifying the third partition of amplified DNA fragments, using primers that are capable of initiating polymerization at the first set of adapters in one end, e.g., 3′-end, and initiating polymerization at the second set of adapters in another end, e.g., 5′-end of the amplified adapter-ligated DNA fragments. In some embodiments, the primers are complementary to at least a portion of the first set of adapters at the 3′-end and are complementary to at least a portion of the complementary sequences of the second set of adapters at the 5′-end of the amplified adapter-ligated DNA fragments.

In some embodiments, (e) further comprises capturing at least a subset of the enriched DNA fragments in one or more targeted genomic regions. The capturing may comprise hybrid capture and optional processing operations, such as amplification. The hybrid capture may comprise hybridization-based targeted capture using the hybrid capture probes to cover one or more restriction enzyme cutting sites in the targeted regions. In some embodiments, a pre-amplification is performed before hybrid capture and a post-amplification is performed after hybrid capture. In some embodiments, the amplification is performed after hybrid capture and the pre-amplification is omitted. In some cases, such as PCR-free library preparation, both the PCR amplification before or after hybrid capture are omitted.

In some embodiments, processing the enriched DNA fragments comprises sequencing of the enriched DNA fragments, such as next-generation sequencing, therefore generating sequencing data. The sequenced data, which may provide the counts of the enriched DNA fragments, can be subject to one or a series of downstream analyses. In some embodiments, (b) is performed using one or more methylation-sensitive restriction enzymes, and determining the methylation status comprises deriving the counts of hyper-methylated DNA fragments in the first partition and the counts of hypo-methylated DNA fragments in the second partition from the sequencing data. In some embodiments, (b) is performed using one or more methylation-dependent restriction enzymes, and determining the methylation status comprises deriving the counts of hypo-methylated DNA fragments in the first partition and the counts of hyper-methylated DNA fragments in the second partition from the sequencing data. In some embodiments, the counts comprise the counts of all sequencing reads in the region of interest. In some embodiments, for example, when the plurality of nucleic acid molecules are subject to conditions sufficient to permit the methylated nucleic acid bases to be distinguishable from the un-methylated nucleic acid bases, the counts of hyper-methylated DNA fragments may comprise the counts of DNA fragments with at least about 50%, or at least about 60%, or at least about 70%, or at least about 80%, or at least about 90%, or about 100% of methylated cytosine residues in CpG dinucleotides of individual DNA fragments, and the counts of hypo-methylated DNA fragments comprise the counts of DNA fragments with at least about 50%, or at least about 60%, or at least about 70%, or at least about 80%, or at least about 90%, about 100% of unmethylated cytosine residues in CpG dinucleotides of individual DNA fragments.

In some embodiments, the hyper or hypomethylation status is indicative of the presence or absence of a disease or risk thereof in the individual. The counts of hyper- and/or hypo-methylated DNA fragments may be inputted as features for a trained single-class classifier or multi-class machine learning classifier or artificial intelligence algorithms to measure or detect or predict the presence or absence of diseases in a subject. In some embodiments, the counts may be pre-processed before being inputted into the classifier. In some embodiments, the pre-processing methods may include but are not limited to, logarithmic transformation, standardization, discretization, feature selection, dimension reduction, or any combination thereof. An exemplary single-class classifier or multi-class classifier may comprise support vector machine, random forest, support vector machine, k-nearest neighbor, na\ddot{v}e Bayes, Gaussian process, decision trees, XGBoost, neural networks, linear and quadratic discrimination analysis, logistic regression, general linear models, or analog of, or any combination thereof. In some cases, the pre-processing methods may include normalization of the counts with counts from reference genome regions without methylation-sensitive restriction enzyme and/or methylation-dependent restriction enzyme digestion sites. In some embodiments, the plurality of nucleic acid molecules comprises cell-free DNA and the disease subjects comprise cancer subjects or subjects at elevated risk for (over the general population) or suspected of having cancer. The measuring for hyper- and hypo-methylation in the methods may be a measure of cancer detection. Detecting cancer from the cell-free DNA of a subject may comprise screening the subject for the presence of cancer, and the screening may occur from routine health care maintenance or for suspicion of the presence of cancer. This screening may lead to further diagnostic tests or interventions, such as for the early detection of cancer. Detecting cancer from the cell-free DNA of a subject may be used to detect minimal residual disease and/or predict the relapse of cancer. A treatment decision may be made based on the status of cancer.

In another aspect, the present disclosure provides kits for practicing any method described herein. In some embodiments, the kit comprises reagents to perform any method described herein.

In another aspect, the present disclosure provides methods of treating a subject. In some embodiments, the method comprises performing one or more of the methods disclosed herein on a biological sample from the subject to determine whether the subject has or does not have a condition sensitive to a clinical intervention. In some embodiments, the subject is administered the clinical intervention if the subject has been determined to have the condition sensitive to the clinical intervention. The subject may have, or be suspected of having, cancer, an infectious disease, or a non-communicable disease. The condition sensitive to a clinical intervention may be a neoplasm, a metastasis, an infection, or an autoimmune disorder. In some embodiments, the clinical intervention comprises an effective amount of a chemotherapy, a radiation therapy, a hormone therapy, a targeted therapy, an immunotherapy, a biologic, a cell therapy, an antibiotic, an anti-viral, radiation, cryoablation, or a combination thereof, and/or a surgical intervention. In some embodiments concerning methods of treatment, the method further comprises processing the enriched DNA fragments to determine the methylation status of the enriched DNA fragments. In some embodiments, the methylation status is indicative of whether the subject has or does not have a condition sensitive to the clinical intervention.

In some embodiments, the method comprises monitoring a response to a first clinical intervention in a subject. In some embodiments, the method comprises performing any method described herein on a biological sample from the subject, wherein the subject has received at least one round of the first clinical intervention. In some embodiments, the method further comprises processing enriched DNA fragments to determine the methylation status of the enriched DNA fragments. In some embodiments, the method further comprises administering an additional round of the first clinical intervention or a second clinical intervention subject, based at least in part on the methylation status of the enriched DNA fragments. In some embodiments, the first clinical intervention comprises an effective amount of a chemotherapy, a radiation therapy, a hormone therapy, a targeted therapy, an immunotherapy, a biologic, a cell therapy, an antibiotic, an anti-viral, radiation, cryoablation, or a combination thereof, and/or a surgical intervention. In some embodiments, the second clinical intervention is different from the first clinical intervention. In some embodiments, the second clinical intervention comprises an effective amount of a chemotherapy, a radiation therapy, a hormone therapy, a targeted therapy, an immunotherapy, a biologic, a cell therapy, an antibiotic, an anti-viral, radiation, cryoablation, or a combination thereof, and/or a surgical intervention, or halting of the first clinical intervention.

In another aspect, the present disclosure provides methods of diagnosing or prognosing a disease in a subject. In some embodiments, the method comprises performing any method described herein to determine whether the subject has the disease or the severity of the disease in the subject. The disease may be a cancer, an infectious disease, or a non-communicable disease.

It is specifically contemplated that any limitation discussed with respect to one aspect of the disclosure may apply to any other aspect of the disclosure. Furthermore, any composition of the disclosure may be used in any method of the invention, and any method of the disclosure may be used to produce or to utilize any composition of the disclosure. Aspects set forth in the Examples are also aspects that may be implemented in the context of aspects discussed elsewhere in a different Example or elsewhere in the application, such as in the Summary, Detailed Description, Claims, and Brief Description of the Drawings.

The foregoing discussion has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific aspects disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure. Additional objects, features, aspects, and advantages of the present invention will be set forth in part in the description which follows, and in part will be obvious from the description or may be learned by practice of the invention. Various aspects of the disclosure will be described in sufficient detail to enable those skilled in the art to practice the invention, and it is to be understood that other aspects may be utilized and that changes may be made without departing from the scope of the invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative aspects, in which the principles of the invention are utilized, and the accompanying drawings (also “Figure” and “FIG.” herein), of which:

FIG. 1 illustrates a flowchart of hyper- and hypo-methylation analysis with background DNA elimination.

FIG. 2 illustrates an example of a method of the present disclosure in which hyper-methylated and hypo-methylated nucleic acid molecules are enriched for analysis.

FIG. 3 illustrates a computer system that is programmed or otherwise configured to implement methods provided herein.

While various aspects of the disclosure have been shown and described herein, it will be obvious to those skilled in the art that such aspects are provided by way of example only. Numerous variations, changes, and substitutions may occur to those skilled in the art without departing from the invention. It should be understood that various alternatives to the aspects of the disclosure described herein may be employed.

### DETAILED DESCRIPTION OF THE INVENTION

### I. Examples of Definitions

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect.

As used herein, the term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that aspects described in the context of the term “comprising” may also be implemented in the context of the term “consisting of” or “consisting essentially of.”

As used herein, the terms “one embodiment,” “an embodiment,” “a particular embodiment,” “a related embodiment,” “a specific embodiment,” “a certain embodiment,” “an additional embodiment,” “a further embodiment”, “one aspect,” “an aspect,” “a particular aspect,” “a related aspect,” “a specific aspect,” “a certain aspect,” “an additional aspect,” “a further aspect”, “certain aspects”, “some aspects” or combinations thereof generally indicate that a particular feature, structure, or characteristic described in connection with the aspect is included in at least one aspect of the present invention. Thus, the appearances of the foregoing phrases in various places throughout this specification are not necessarily all referring to the same aspect. Furthermore, the particular features, structures, or characteristics may be combined in any suitable manner in one or more aspects.

A variety of aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, generally refers to an individual having a biological sample that is undergoing processing or analysis. A subject can be an animal or plant. The subject can be a mammal, such as a human, dog, cat, horse, pig or rodent. The subject can be a patient, e.g., have or be suspected of having or at risk (e.g., elevated risk) for having a disease, such as one or more cancers (e.g., brain cancer, breast cancer, cervical cancer, colorectal cancer, endometrial cancer, esophageal cancer, gastric cancer, hepatobiliary tract cancer, leukemia, liver cancer, lung cancer, lymphoma, ovarian cancer, pancreatic cancer, skin cancer, urinary tract cancer, testicular cancer, kidney cancer, sarcoma, bile duct cancer, thyroid cancer, gall bladder cancer, spleen cancer, or prostate cancer, and cancer may or may not comprise solid tumor(s)), one or more infectious diseases, one or more genetic disorders, or one or more tumors, or any combination thereof. For subjects having or suspected of having one or more tumors, the tumors may be of one or more types. The subject may have a disease or be suspected of having the disease. The subject may be asymptomatic. The subject may be at risk of the disease, such as at an elevated risk greater than the general population.

The term “sample,” as used herein, generally refers to a biological sample. The samples may be taken from tissue and/or cells or from the environment of tissue and/or cells and/or circulatory system. In some examples, the sample may comprise, or be derived from, a tissue biopsy, blood (e.g., whole blood), blood plasma, serum, bone marrow, cerebral spinal fluid, pleural fluid, saliva, stool, urine, extracellular fluid, dried blood spots, cultured cells, culture media, discarded tissue, plant matter, synthetic proteins, bacterial and/or viral samples, fungal tissue, archaea, or protozoans. The sample may have been isolated from the source prior to collection. Samples may comprise forensic evidence. Non-limiting examples include a fingerprint, saliva, urine, blood, stool, semen, or other bodily fluids isolated from the primary source prior to collection. In some examples, the sample is isolated from its primary source (cells, tissue, bodily fluids such as blood, environmental samples, etc.) during sample preparation. The sample may be derived from an extinct species including but not limited to samples derived from fossils. The sample may or may not be purified or otherwise enriched from its primary source. In some cases the primary source is homogenized prior to further processing. The sample may be filtered or centrifuged to remove buffy coat, lipids, or particulate matter. The sample may also be purified or enriched for nucleic acids, or may be treated with RNases or Dnases. The sample may contain tissues and/or cells that are intact, fragmented, or partially degraded.

The sample may be obtained from a subject with a disease or disorder, a subject suspected of having a disease or disorder, and/or a subject who may or may not have had a diagnosis of the disease or disorder. The subject may be in need of a second opinion. The disease or disorder may be an infectious disease, an immune disorder or disease, a cancer, a genetic disease, a degenerative disease, a lifestyle disease, or an injury. The infectious disease may be caused by bacteria, viruses, fungi, and/or parasites. Non-limiting examples of cancers include pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, thyroid cancer, gall bladder cancer, spleen cancer, and prostate cancer. Some examples of genetic diseases or disorders include, but are not limited to, cystic fibrosis, Charcot-Marie-Tooth disease, Huntington's disease, Peutz-Jeghers syndrome, Down syndrome, Rheumatoid arthritis, and Tay-Sachs disease. Non-limiting examples of lifestyle diseases include obesity, diabetes, arteriosclerosis, heart disease, stroke, hypertension, liver cirrhosis, nephritis, cancer, chronic obstructive pulmonary disease (COPD), hearing problems, and chronic backache. Some examples of injuries include, but are not limited to, abrasion, brain injuries, bruising, burns, concussions, congestive heart failure, construction injuries, dislocation, flail chest, fracture, hemothorax, herniated disc, hip pointer, hypothermia, lacerations, pinched nerve, pneumothorax, rib fracture, sciatica, spinal cord injury, tendons ligaments fascia injury, traumatic brain injury, and whiplash. The sample may be taken before and/or after treatment of a subject with a disease or disorder. Samples may be taken before and/or after a treatment of the subject for a disease or disorder. Samples may be taken during a treatment or a treatment regimen. Multiple samples may be taken from a subject to monitor the effects of a treatment over time, including beginning from prior to the onset of the treatment. The sample may be taken from a subject known or suspected of having an infectious disease for which diagnostic reagents, such as antibodies, may or may not be available. Samples may be taken from a subject to monitor abnormal tissue-specific cell death or organ transplantation.

The sample may be taken from a subject having or suspected of having a disease or a disorder. The sample may be taken from a subject experiencing unexplained symptoms, such as fatigue, nausea, weight loss, aches, pains, weakness, abnormal growth(s), or memory loss. The sample may be taken from a subject having explained symptoms. The sample may be taken from a subject at elevated risk of developing a disease or disorder because of one or more factors such as familial and/or personal history, age, environmental exposure, lifestyle risk factors, presence of other known risk factor(s), or a combination thereof.

The sample may be taken from a healthy individual. In some cases, samples may be taken longitudinally from the same individual. In some cases, samples acquired longitudinally may be analyzed with the goal of monitoring individual health and early detection of health issues (e.g., early diagnosis of cancer). In some aspects, the sample may be collected at a home setting or at a point-of-care setting and subsequently transported by a mail delivery, courier delivery, or other transport method prior to analysis. For example, a home user may collect a blood spot sample through a finger prick, and the blood spot sample may be dried and subsequently transported by mail delivery prior to analysis. In some cases, samples acquired longitudinally may be used to monitor response to stimuli expected to impact health, athletic performance, or cognitive performance. Non-limiting examples include response to medication, dieting, and/or an exercise regimen. In some cases, the individual sample is multi-purpose and allows for hyper-/hypo-methylated profiling to obtain clinically relevant information but also is used for information about the individual's personal or family ancestry. In some cases, the samples may be collected from a pregnant woman and/or her fetus.

A biological sample may be a nucleic acid sample including one or more nucleic acid molecules. The nucleic acid molecules may be cell-free or substantially cell-free nucleic acid molecules, such as cell-free DNA (cfDNA) or cell-free RNA (cfRNA) or a mixture thereof. The nucleic acid molecules may be derived from a variety of sources including human, mammal, non-human mammal, ape, monkey, chimpanzee, reptilian, amphibian, or avian sources. Further, samples may be extracted from variety of animal fluids containing cell-free sequences, including but not limited to blood, serum, plasma, bone marrow, vitreous, sputum, stool, urine, tears, perspiration, saliva, semen, mucosal excretions, mucus, cerebral spinal fluid, pleural fluid, amniotic fluid, and lymph fluid. The sample may be taken from an embryo, fetus, or pregnant woman. In some examples, the sample may be isolated from the mother's blood plasma. In some examples, the sample may comprise cell-free nucleic acids (e.g., cfDNA) that are fetal in origin (via a bodily sample obtained from a pregnant subject), or are derived from tissue of the subject itself.

Components of the sample (including nucleic acids) may be tagged, e.g., with identifiable tags, to allow for identifying of detecting or multiplexing of samples. Some non-limiting examples of identifiable tags include: fluorophores, magnetic nanoparticles, and nucleic acid barcodes. Fluorophores may include fluorescent proteins such as GFP, YFP, RFP, eGFP, mCherry, tdtomato, FITC, Alexa Fluor 350, Alexa Fluor 305, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Pacific Blue, Coumarin, BODIPY FL, Pacific Green, Oregon Green, Cy3, Cy5, Pacific Orange, TRITC, Texas Red, Phycoerythrin, Allophcocyanin, or other fluorophores. The intensity of fluorescence signal can be used to quantitate the abundance of nucleic acid molecules in the sample, or to determine the presence or absence of nucleic acid molecules in the sample. One or more barcode tags may be attached (e.g., by coupling or ligating) to cell-free nucleic acids (e.g., cfDNA) in the sample prior to sequencing. The barcodes may uniquely tag the cfDNA molecules in a sample. Alternatively, the barcodes may non-uniquely tag the cfDNA molecules in a sample. The barcode(s) may non-uniquely tag the cfDNA molecules in a sample such that additional information taken from the cfDNA molecule (e.g., at least a portion of the endogenous sequence of the cfDNA molecule), taken in combination with the non-unique tag, may function as a unique identifier for (e.g., to uniquely identify against other molecules) the cfDNA molecule in a sample. For example, cfDNA sequence reads having unique identity (e.g., from a given template molecule) may be detected based on sequence information comprising one or more contiguous-base regions at one or both ends of the sequence read, the length of the sequence read, and the sequence of the attached barcodes at one or both ends of the sequence read. DNA molecules may be uniquely identified without tagging by partitioning a DNA (e.g., cfDNA) sample into many (e.g., at least about 50, at least about 100, at least about 500, at least about 1 thousand, at least about 5 thousand, at least about 10 thousand, at least about 50 thousand, or at least about 100 thousand) different discrete subunits (e.g., partitions, wells, or droplets) prior to amplification, such that amplified DNA molecules can be uniquely resolved and identified as originating from their respective individual input molecules of DNA.

Any number of samples may be multiplexed. For example, a multiplexed analysis may contain at least about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, or more samples. The identifiable tags may provide a way to interrogate each sample as to its origin, or may direct different samples to segregate to different areas or a solid support.

Any number of samples may be mixed prior to analysis without tagging or multiplexing. For example, a multiplexed analysis may contain at least about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, or more samples. Samples may be multiplexed without tagging using a combinatorial pooling design in which samples are mixed into pools in a manner that allows signal from individual samples to be resolved from the analyzed pools using computational demultiplexing.

The samples may be enriched prior to sequencing. For example, the cfDNA molecules may be selectively enriched or non-selectively enriched for one or more regions from the subject's genome or transcriptome. For example, the cfDNA molecules may be selectively enriched for one or more regions from the subject's genome or transcriptome by targeted sequence capture (e.g., using a panel), selective amplification, and/or targeted amplification (e.g., targeted polymerase chain reaction (PCR)). As another example, the cfDNA molecules may be non-selectively enriched for one or more regions from the subject's genome or transcriptome by universal amplification (e.g., universal PCR). In some aspects, amplification comprises universal amplification, whole genome amplification, or non-selective amplification. The cfDNA molecules may be size selected for fragments having a length in a predetermined range. For example, size selection can be performed on DNA fragments prior to adapter ligation for lengths in a range of about 40 base pairs (bp) to about 250 bp. Specific ranges include 40-250, 40-200, 40-150, 40-100, 50-250, 50-200, 50-150, 50-100, 100-250, 100-200, 100-150, 150-250, 150-200, or 175-200 bp. As another example, size selection can be performed on DNA fragments after adapter ligation for lengths in a range of about 160 bp to about 400 bp. Specific ranges include 160-400, 160-300, 160-200, 175-400, 175-300, 175-200, 200-400, 200-300, or 300-400 bp.

The term “nucleic acid,” or “polynucleotide,” as used herein, generally refers to a molecule comprising one or more nucleic acid subunits, or nucleotides. A nucleic acid may include one or more nucleotides selected from adenosine (A), cytosine (C), guanine (G), thymine (T), and uracil (U), or variants thereof. A nucleotide generally includes a nucleoside and at least 1, 2, 3, 4, 5, 6, 7, 8, 9, 10, or more phosphate (PO3) groups. A nucleotide can include a nucleobase, a five-carbon sugar (either ribose or deoxyribose), and one or more phosphate groups, individually or in combination.

The terms “nucleic acid molecule,” “nucleic acid sequence,” “nucleic acid fragment,” “oligonucleotide” and “polynucleotide,” as used herein, generally refer to a polynucleotide, such as deoxyribonucleotides (DNA) or ribonucleotides (RNA), or analogs and/or combinations thereof (e.g., mixture of DNA and RNA). A nucleic acid molecule may have various lengths. A nucleic acid molecule can have a length of at least about 5 bases, 10 bases, 20 bases, 30 bases, 40 bases, 50 bases, 60 bases, 70 bases, 80 bases, 90, 100 bases, 110 bases, 120 bases, 130 bases, 140 bases, 150 bases, 160 bases, 170 bases, 180 bases, 190 bases, 200 bases, 300 bases, 400 bases, 500 bases, 1 kilobase (kb), 2 kb, 3, kb, 4 kb, 5 kb, 10 kb, or 50 kb, or it may have any number of bases between any two of the aforementioned values. An oligonucleotide may comprise a specific sequence of four nucleotide bases: adenine (A); cytosine (C); guanine (G); and thymine (T) (uracil (U) for thymine (T) when the polynucleotide is RNA). Thus, the terms “nucleic acid molecule,” “nucleic acid sequence,” “nucleic acid fragment,” “oligonucleotide” and “polynucleotide” are at least in part intended to be the alphabetical representation of a polynucleotide molecule. Alternatively, the terms may be applied to the polynucleotide molecule itself. This alphabetical representation can be input into databases in a computer having a central processing unit and/or used for bioinformatics applications such as functional genomics and homology searching. Oligonucleotides may include one or more nonstandard nucleotide(s), nucleotide analog(s) and/or modified nucleotides.

The term “probe,” as used herein, generally refers to a nucleotide sequence to which nucleic acids from a sample can hybridize. Probes specifically bind to a targeted nucleotide sequence of complementary, substantially complementary, or partially complementary. In some aspects, the probe is labeled. In some aspects, the label on the probe is fluorescent label designed for detection. In some aspects, the label on the probe comprises biotinylation of one or more nucleotide.

The term “methylation status,” as used herein, generally refers to the methylation, unmethylation, hyper methylation, and hypo methylation status of cytosine residues in CpG dinucleotides.

The term “hyper methylation,” as used herein, refers to methylation of at least about 50%, or at least about 60%, or at least about 70%, or at least about 80%, or at least about 90%, or 100% of cytosine residues in CpG dinucleotides of the nucleic acid molecule. The term “hypo methylation,” as used herein, refers to unmethylation of least about 50%, or at least about 60%, or at least about 70%, or at least about 80%, or at least about 90%, or 100% of cytosine residues in CpG dinucleotides of the nucleic acid molecule.

The term “target regions,” as used herein, generally refers to a nucleotide sequence in the genome that has different methylation status in nucleic acid molecules of interest, e.g., tumor-derived cell-free DNA has different methylation status from the background nucleic acid molecules. The hyper- or hypo-methylation of nucleic acid molecules in these regions may be indicative of the presence or absence, respectively, of a disease or disorder in the subject.

The term “nucleic acid molecules of interest,” as used herein, generally refers to diseased associated nucleic acid molecules. For example, for cancer detection using cell-free DNA, the nucleic acid molecules of interest may refer to DNA released from tumor cells.

The terms “cell-free DNA” or “cfDNA,” as used herein, generally refer to DNA that is freely circulating in fluids of a body, such as the bloodstream or plasma therefrom. In some aspects of methods utilized herein, the cfDNA encompasses a particular type of cfDNA, such as circulating tumor DNA (ctDNA) that is tumor-derived fragmented DNA in the bloodstream that is not associated with cells. The cfDNA may be double-stranded, single-stranded, or have characteristics of both.

The terms “clinical intervention” and “therapeutic intervention” may be used interchangeably and can refer to compositions and/or methods for treating an individual.

### II. Examples of Methods of the Disclosure and Compositions Thereof

The present disclosure provides methods and systems for hyper-methylated and/or hypo-methylation analysis by enriching for either or both hyper-methylated and hypo-methylated nucleic acid molecules of interest from hypo-methylated and/or hyper-methylated background nucleic acid molecules in a mixture of nucleic acid molecules. Aspects of the disclosure include methods that employ a series of operations to produce sequencing libraries of hyper-methylated and/or hypo-methylated DNA fragments. In particular aspects, the methods comprise ligating the first adapters to the ends of DNA fragments, digesting the adapter-ligated DNA fragments with methylation-sensitive or methylation-dependent restriction enzymes, ligating the second adapters to the digested DNA fragments, amplifying DNA fragments with adapters on the ends, partitioning the amplified DNA fragments, enriching hyper- or hypo-methylated DNA fragments from different partitions, processing the enriched DNA fragments for hyper- and hypo-methylation analysis. In particular aspects, the source of nucleic acid molecules from which the libraries are generated includes DNA of any kind, particularly cell-free DNA (cfDNA). In particular aspects, the libraries are generated following DNA fragmentation using different methods, for example, sonication with shearing devices and/or digestion with restriction enzymes. In some cases the starting nucleic acid material itself may comprise fragments (such as fragmentation of a natural source (from cell apoptosis or necrosis, including from cancer cell DNA)).

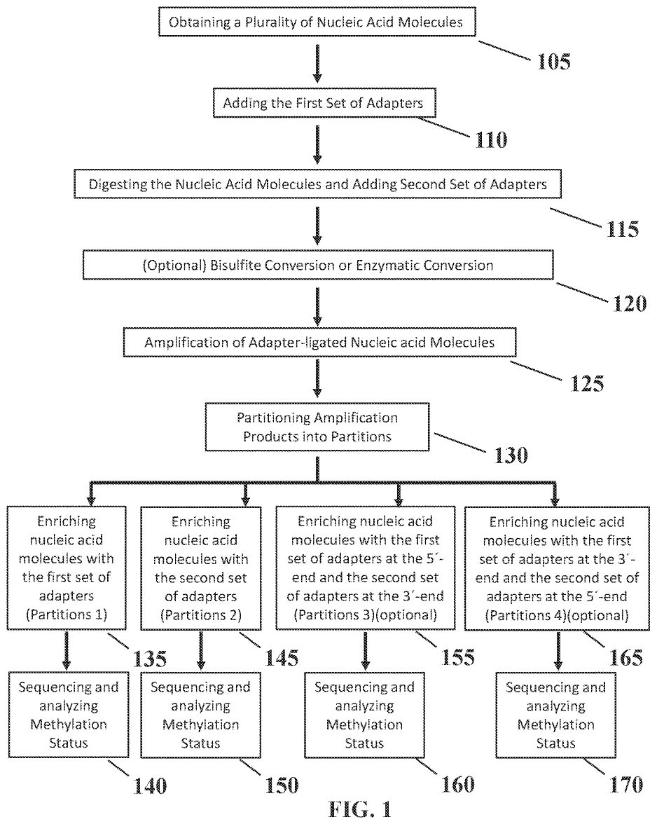

FIG. 1 illustrates a flowchart of an example of applying the described method of hyper-methylated and/or hypo-methylation analysis with background elimination. In operation 105, nucleic acid molecules, e.g., cfDNA, may be obtained from a subject to be tested. In operation 110, the first set of adapters is ligated to the end of nucleic acid molecules. In operation 115, the adapter-ligated nucleic acid molecules are subjected to methylation-sensitive or methylation-dependent restriction enzyme digestion. For methylation-sensitive digestion, the adapter-ligated nucleic acid molecules with un-methylated cutting sites may be digested so that the digested nucleic acid molecule has one or none of the first set of adapters on its ends. For methylation-dependent digestion, the adapter-ligated nucleic acid molecules with methylated cutting sites may be digested so that the digested nucleic acid molecule has one or none of the first set of adapters on its ends. The digestion may create a digestion-enzyme-specific overhang on the end of the digested nucleic acid molecule, e.g., 5′-CG overhang in the nucleic acid molecule digested by HpaII enzyme. A second set of adapters that have overhangs that complement the digestion-enzyme-specific 5′- or 3′-overhangs may be ligated to the digested nucleic acid molecules. The second set of adapters is distinguishable by sequencer from that of the first set of adapters. The digestion and ligation of the second set of adapters can be performed in the same reaction or different reactions. For methylation-sensitive digestion, only nucleic acid molecules with methylated restriction enzyme cutting sites or not any restriction enzyme cutting sites may have the first set of adapters on both ends, and only nucleic acid molecules with two un-methylated restriction enzyme cutting sites may have the second set of adapters on both ends. For methylation-dependent digestion, only nucleic acid molecules with un-methylated restriction enzyme cutting sites or not any restriction enzyme cutting sites may have the first set of adapters on both ends, and only nucleic acid molecules with two methylated restriction enzyme cutting sites may have the second set of adapters on both ends.

In operation 120 (optional), the nucleic acid molecules may be optionally subject to conditions to permit the methylated nucleic acid bases to be distinguishable from the unmethylated nucleic acid bases, for example, bisulfite conversion and enzymatic conversion.

In operation 125, after adding the first and second set of adapters, the adapter-ligated nucleic acid molecules are then subject to PCR amplification with a limited number of PCR cycles. The primers for PCR reaction are designed to recognize both the first set of adapters and the second set of adapters. The limited number of PCR cycles are used to create multiple copies of nucleic acid molecules so that each partition in the partitioning step has at least one or more copies of the nucleic acid molecules. Next, in operation 130, the amplified nucleic acid molecules are partitioned (e.g., in this exemplary flowchart, the amplified nucleic acid molecules are partitioned to four partitions) for further analysis.

In operation 135, nucleic acid molecules with the first set of adapters on both ends are enriched. The enrichment may be performed by PCR amplification utilizing primers that are capable of initiating polymerization at the first set of adapters. The PCR amplification may be followed by hybridization-based targeted capture using the probes to cover one or more restriction enzyme cutting sites in the targeted regions. In operation 140, the enriched nucleic acid molecules are sequenced using one of the sequencers, e.g., Illumina HiSeq 2000. Bioinformatics analysis is performed to determine the counts and methylation status of the nucleic acid molecules. For the use of methylation-sensitive restriction enzymes in the digestion step, nucleic acid molecules with one or more un-methylated restriction enzyme cutting sites are digested and the digested nucleic acid molecules have the first set of adapters on one or not any of the ends, thus cannot be enriched from Partition 1. The counts of nucleic molecules represent hyper-methylated nucleic acid molecules. For the use of methylation-dependent restriction enzymes in the digestion operation, nucleic acid molecules with one or more methylated restriction enzyme cutting sites are digested and the digested nucleic acid molecules have the first set of adapters on one or not any of the ends, thus cannot be enriched from Partition 1. The counts of nucleic molecules represent hypo-methylated nucleic acid molecules.

In operation 145, nucleic acid molecules with the second set of adapters on both ends are enriched. The enrichment may be performed by PCR amplification utilizing primers that are capable of initiating polymerization at the second set of adapters. The PCR amplification may or may not be followed by hybridization-based targeted capture using the probes to cover one or more restriction enzyme cutting sites in the targeted regions. In operation 150, the enriched nucleic acid molecules are sequenced using one of the sequencers, e.g., Illumina HiSeq 2000. Bioinformatics analysis is performed to determine the counts and methylation status of the nucleic acid molecules. For the use of methylation-sensitive restriction enzymes in the digestion step, only nucleic acid molecules with two or more un-methylated restriction enzyme cutting sites can have the second set of adapters on both ends of the digested fragments, thus can be enriched from Partition 2. The counts of nucleic molecules represent hypo-methylated nucleic acid molecules. For the use of methylation-dependent restriction enzymes in the digestion operation, only nucleic acid molecules with two or more methylated restriction enzyme cutting sites can have the second set of adapters on both ends of the digested fragments, thus can be enriched from Partition 2The counts of nucleic molecules represent hyper-methylated nucleic acid molecules.

In operation 155, nucleic acid molecules with the first set of adapters at the 5′-end and the second set of adapters at the 3′-end of amplified adapter-ligated nucleic acid molecules are enriched from Partition 3. The enrichment may be performed by PCR amplification utilizing primers that are capable of initiating polymerization at the second set of adapters at the 3′-end and initiating polymerization at the first set of adapters at the 5′-end of the amplified adapter-ligated DNA fragments. The primers are complementary to at least a portion of the second set of adapters at the 3′-end and are complementary to at least a portion of the complementary sequences of the first set of adapters at the 5′-end of the amplified adapter-ligated DNA fragments. The PCR amplification may or may not be followed by hybridization-based targeted capture using the probes to cover one or more restriction enzyme cutting sites in the targeted regions. In operation 160, the enriched nucleic acid molecules are sequenced using one of the sequencers, e.g., Illumina HiSeq 2000. Bioinformatics analysis is performed to determine the counts and methylation status of the nucleic acid molecules.

In operation 165, nucleic acid molecules with the first set of adapters at the 3′-end and the second set of adapters at the 5′-end of amplified adapter-ligated nucleic acid molecules are enriched from Partition 4. The enrichment may be performed by PCR amplification utilizing primers that are capable of initiating polymerization at the first set of adapters at the 3′-end and initiating polymerization at the second set of adapters at the 5′-end of the amplified adapter-ligated DNA fragments. The primers are complementary to at least a portion of the first set of adapters at the 3′-end and are complementary to at least a portion of the complementary sequences of the second set of adapters at the 5′-end of the amplified adapter-ligated DNA fragments. The PCR amplification may or may not be followed by hybridization-based targeted capture using the probes to cover one or more restriction enzyme cutting sites in the targeted regions. In operation 170, the enriched nucleic acid molecules are sequenced using one of the sequencers, e.g., Illumina HiSeq 2000. Bioinformatics analysis is performed to determine the counts and methylation status of the nucleic acid molecules.

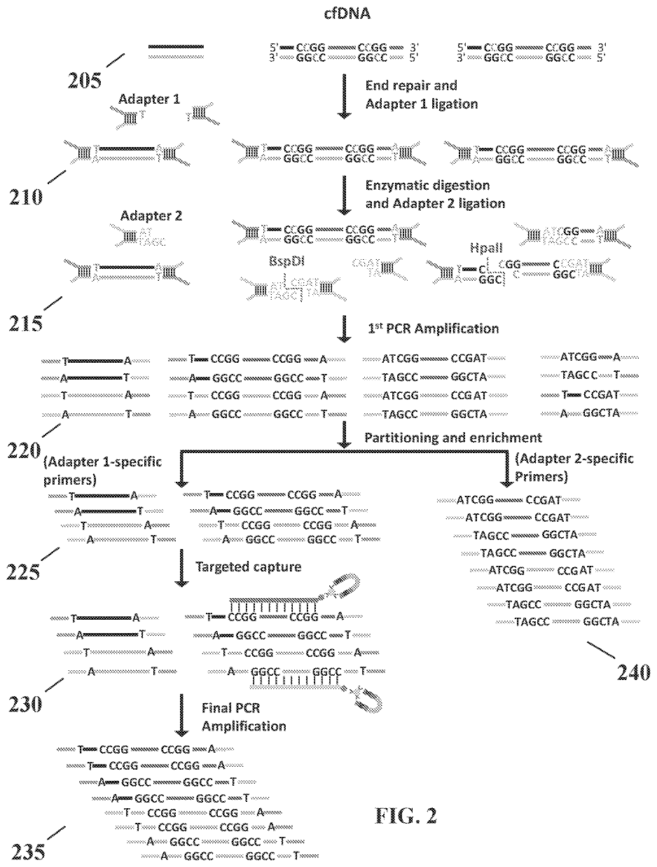

FIG. 2 illustrates an example of enriching hyper-methylated and hypo-methylated cell-free DNA molecules for methylation analysis for applications, such as cancer diagnosis. Operation 205 provides a mixture of types of DNA molecules in cfDNA. In this example, in operation 210, Adapter 1 may be ligated to the cfDNA molecules. Prior to adapter ligation, DNA end repair (3′-end blunting and/or 3′-end A-tailing) and 5′-end phosphorylation reactions may be performed. These adapter-ligated cfDNA molecules are subjected to digestion by one or more restriction enzymes. In this example, methylation-sensitive restriction enzyme HpaII is used in operation 215, and Adapter 2 is added in the same reaction as HpaII digestion. The use of Hpall cuts adapter-ligated cfDNA molecules containing CCGG recognition site where the second cytosine residue in the recognition site is un-methylated (colored in blue to indicate un-methylated C and in red to indicate methylated C in FIG. 2). The designed Adapter 2 contains 3′-CG overhangs that can ligate to the Hpall cleaved ends on the nucleic acid molecules. In the example of FIG. 2, the adapters can form adapter dimers with 5′-ATCGAT-3′ sequence at the junction of adapter dimers. However, the junction between an adapter and the ends of Hpall-cleaved cell-free DNA molecules comprises different sequences: 5′-ATCGG-3′. To avoid adapter-adapter ligation to form adapter dimer production, a restriction enzyme BspDI is added into the reaction that can recognize and cut the adapter-adapter junction, whereas Hpall in the mixture can digest the ligated cell-free DNA molecules. The second set of adapters is distinguishable by sequencer from that of the first set of adapters. In this example, the Adapter 1 is compatible with the Illumina Nextera adapter and Adapter 2 is compatible with the Illumina TruSeq adapters.

In operation 220, the adapter-ligated cfDNA molecules are subject to a few cycles, e.g., 1, 2, 3, 4, or 5 cycles, of PCR amplification. Multiple copies of adapter-ligated cfDNA molecules are generated. The adapter-ligated cfDNA molecules are not subject to conditions to permit the methylated nucleic acid bases to be distinguishable from the unmethylated nucleic acid bases, for example, bisulfite conversion and enzymatic conversion, prior to the PCR amplification. The amplified cfDNA molecules are then partitioned into two partitions. In operation 225, the first partition is amplified with Adapter-1-specific primers that are capable of initiating polymerization at the first set of adapters. Next, in operation 230, the amplified cfDNA molecules are hybridized to probes that are complementary or substantially complementary to at least a portion of cfDNA molecules in the targeted regions with a high level of methylation, for example, at least about 90% of cytosine residues in CpG dinucleotides of the nucleic acid molecules are methylated. One or more nucleotides in the probe may be biotinylated. The captured DNA fragments may be subjected to post-amplification, such as using polymerase chain reaction (PCR), followed by nucleic acid sequencing in operation 235. The sequence reads from the first partition represent hyper-methylated cfDNA fragments with the elimination of hypo-methylated cfDNA fragments. In operation 240, the second partition is amplified with Adapter-2-specific primers that are capable of initiating polymerization at the second set of adapters and followed by nucleic acid sequencing. cfDNA fragments from the second partition have unmethylated Hpall cutting sites on both of their ends. Given the pervasive feature of methylation, those cfDNA fragments are likely hypomethylated in other CpG sites as well. Thus, the sequence reads from the second partition represent hypo-methylated cfDNA fragments with the elimination of hyper-methylated cfDNA fragments.

The method disclosed herein may be implemented in a diagnostic test that encompasses reagents and a machine-learning classifier. The reagents may include the capture substrates, methylation-sensitive restriction enzymes and/or methylation-dependent restriction enzymes, adapters, and polymerase. Sequencing library may be prepared and analyzed utilizing any of the methods of disclosure following the instruction in the test. The counts of hyper-methylated and hypo-methylated nucleic acid molecules may be inputted as features for the classifier, generating a likelihood of a subject as having or being suspected of having a disease or disorder.

### III. Nucleic Acid Molecules for Hyper-/Hypo-Methylation Analysis

Hyper-/Hypo-methylation analysis may be performed on nucleic acid molecules, such as DNA or RNA. In particular aspects, the nucleic acid molecules from which the hyper-/hypo-methylation analysis is prepared is DNA, and the DNA in some cases is cell-free DNA (cfDNA). The cfDNA may be obtained from an individual, including a mammal. The cfDNA may be from an individual in need of analysis of the cfDNA, for example to provide a determination concerning their health, such as detecting a disease condition or risk or susceptibility thereto. The cfDNA may be from one or more samples from the individual. The sample may be from plasma, blood, serum, bone marrow, cerebral spinal fluid, pleural fluid, saliva, stool, or urine, in some cases. The cfDNA from which the hyper-/hypo-methylation analysis is prepared may be double-stranded, single-stranded, or a mixture thereof.

In some aspects, the nucleic acid molecules for which a hyper-/hypo-methylation analysis is desired to be performed may be modified prior to utilization in methods of the disclosure. For example, the nucleic acid molecules may be enriched for a certain type of nucleic acid molecule, a certain size of nucleic acid molecules, or a combination thereof. In particular cases, the nucleic acid molecules are cfDNA that has been enriched, for example for a certain size of molecule.

### IV. Applications of Hyper-/Hypo-Methylation Analysis

Cancer cells may display aberrant DNA methylation patterns. Hypermethylated and/or hypomethylated tumor DNA fragments can be released into the bloodstream via processes such as cell apoptosis or necrosis, where they may become part of circulating cell-free DNA (cfDNA) in bodily fluids such as plasma or urine. Such cfDNA may be subjected to methylation profiling for clinical diagnostic applications such as cancer screening. The minimally invasive or non-invasive nature of cfDNA methylation profiling may render such cfDNA methylation profiling an effective strategy for general cancer screening or cancer diagnosis, prognosis, treatment selection, or treatment monitoring. For example, whole-genome bisulfite sequencing can provide a comprehensive view of the DNA methylome, but can be expensive to deep sequence the entire genome.

Certain aspects of the disclosure concern methods, systems, and compositions related to analysis of the counts of hyper-/hypo-methylated nucleic acid molecules, for the purpose of measuring or detecting or determining a presence or absence of a disease or disorder, and so forth. In particular aspects, the molecules comprise cfDNA, and in some aspects the cfDNA is from an individual (such as blood or plasma or urine (or a combination thereof) samples from the individual).

For aspects of the disclosure related to disease, such as cancer, analysis of cfDNA in suitable samples can be an effective method for obtaining information. For example, following hyper-/hypo-methylation analysis, the counts of hyper-/hypo-methylated nucleic acid molecules in the targeted region may be utilized for determining if an individual has a particular disease or medical condition or is at elevated risk for or susceptibility thereof. In an example, the individual has or is suspected of having or is at elevated risk of having cancer, and the hyper-/hypo-methylation analysis of prepared cfDNA molecules assists in determining whether the individual has or is suspected of having or is at elevated risk of having cancer.

In some aspects, the hyper-/hypo-methylation analysis methods involve non-invasive cancer screening, including identifying the tumor tissue-of-origin. Liquid biopsy (which may also be referred to as fluid biopsy or fluid phase biopsy), e.g., blood draw, unlike traditional tissue biopsy, is useful for identifying a variety of different malignancies and may be utilized in methods encompassed in the disclosure.

In some aspects, a plurality of cfDNA molecules is obtained from a bodily sample of the subject. In some aspects, the bodily sample is selected from the group consisting of plasma, serum, bone marrow, cerebral spinal fluid, pleural fluid, saliva, stool, sputum, nipple aspirate, biopsy, cheek scrapings, urine, and a combination thereof. In some aspects, the method further comprises identifying molecules having hyper-/hypo-methylation in the targeted regions to obtain their counts (e.g. only count those with certain methylation patterns). In some aspects, the method further comprises processing the counts of hyper-/hypo-methylated cfDNA molecule in the targeted regions to generate a likelihood of the subject as having or being suspected of having a disease or disorder.

In some aspects, the disease or disorder for which information is desired is selected from the group consisting of cancer, multiple sclerosis, traumatic or ischemic brain damage, diabetes, pancreatitis, Alzheimer's disease, and fetal abnormality. In some aspects, the disease or disorder is a cancer selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, thyroid cancer, gall bladder cancer, spleen cancer, and prostate cancer.

In some aspects, hyper-/hypo-methylation analysis of cfDNA molecules, obtained from a bodily sample of the subject, can be used to monitor abnormal tissue-specific cell death or organ transplantation.

In some aspects, cfDNA hyper-/hypo-methylation analysis can be used to diagnose a patient who has symptoms of cancer, is asymptomatic of cancer, has a family or patient history of cancer, is at elevated risk for cancer, or who has been diagnosed with cancer. A subject may be a mammalian subject, such as a human subject. The cancer may be malignant, benign, metastatic, or a precancer. In still further aspects, the cancer is melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, liver, mesothelioma, cervical, gastrointestinal, lymphoma, brain, colon, sarcoma, gall bladder thyroid, spleen, or bladder. The cancer may include a tumor comprised of tumor cells.

In some aspects, there are methods for treating cancer in a subject (e.g., cancer patient) following determination of a need thereof based on methods and systems herein of hyper-/hypo-methylation analysis. Such methods of treating may comprise administering to the patient an effective amount of chemotherapy, radiation therapy, hormone therapy, targeted therapy, or immunotherapy (or a combination thereof) after the patient has been determined to have cancer based on methods disclosed herein. The point of origin of the cancer may be determined, in which case, the treatment is tailored to cancer of that origin. In some aspects, tumor resection is performed as the treatment or may be part of the treatment with one of the other treatments. Examples of chemotherapeutics include, but are not limited to: alkylating agents such as bifunctional alkylators (for example, cyclophosphamide, mechlorethamine, chlorambucil, melphalan) or monofunctional alkylators (for example, dacarbazine (DTIC), nitrosoureas, temozolomide (oral dacarbazine)); anthracyclines (for example, daunorubicin, doxorubicin, epirubicin, idarubicin, mitoxantrone, and valrubicin; taxanes, which disrupt the cytoskeleton (for example, paclitaxel, docetaxel, abraxane, taxotere); epothilones; histone deacetylase inhibitors (for example, vorinostat, romidepsin); Topoisomerase I inhibitors (for example, irinotecan, topotecan); Topoisomerase II inhibitors (for example, etoposide, teniposide, tafluposide); kinase inhibitors (for example, bortezomib, erlotinib, gefitinib, imatinib, vemurafenib, and vismodegib); nucleotide analogs and nucleotide precursor analogs (for example, azacitidine. Azathioprine, capecitabine, cytarabine, doxifluridine. Fluorouracil, gemcitabine, hydroxyurea, mercaptopurine, methotrexate, tioguanine (formerly thioguanine); peptide antibiotics (for examples, bleomycin, actinomycin); platinum-based antineoplastics (for example, carboplatin, cisplatin, oxaliplatin); retinoids (for example, retinoin, alitretinoin, bexarotene); and vinca alkaloids (for example, vinblastine, vincristine, vindesine, and vinorelbine). Examples of immunotherapies include, but are not limited to, cellular therapy such as dendritic cell therapy (for example, involving chimeric antigen receptor); antibody therapy (for example, Alemtuzumab, Atezolizumab, Ipilimumab, Nivolumab, Ofatumumab, Pembrolizumab, Rituximab or other antibodies with the same target as one of these antibodies, such as CTLA-4, PD-1, PD-L1, or other checkpoint inhibitors); and cytokine therapy (for example, interferon or interleukin).

In some aspects, methods of using cfDNA hyper-/hypo-methylation analysis to diagnose a subject may further involve performing a biopsy, acquiring a computerized tomography scan (CT or CAT) scan, acquiring a positron emission tomography (PET) scan, acquiring a magnetic resonance imaging (MRI) scan, acquiring a mammogram, acquiring an ultrasound scan, or otherwise evaluating tissue suspected of being cancerous before or after the patient's cfDNA hyper-/hypo-methylation analysis. In some aspects, cancer that is detected is classified in a cancer classification or staging (e.g., stage I, stage II, stage III, or stage IV).

In some aspects, cfDNA hyper-/hypo-methylation analysis by methods and systems disclosed herein is utilized for monitoring a therapy and/or monitoring tumor progression, including during and/or after treatment. For example, blood draws may be obtained from a subject at various time points to monitor tumor progression throughout one or more treatment regimens, and the cfDNA therefrom may be assayed.

In some aspects, cfDNA hyper-/hypo-methylation analysis by methods and systems of the present disclosure may be utilized for assessment of disease stage or as a prognostic biomarker, for example in cases where a tissue biopsy is not possible or where archived tumor samples are not available for genetic analysis.

In some aspects, cfDNA hyper-/hypo-methylation analysis by methods and systems provided herein may be used for screening and early detection of cancer. For example, blood draws may be obtained regularly from an individual without any symptoms of cancer to find cancer early or to ascertain a predisposition to cancer.

In some aspects, cfDNA hyper-/hypo-methylation analysis by methods and systems provided herein may be used for prenatal testing of fetal DNA from maternal plasma or serum for identification of Down syndrome and other chromosomal abnormalities in a fetus.

In some aspects, cfDNA hyper-/hypo-methylation analysis obtained by methods and systems provided herein may be used for organ transplantation monitoring.

In some aspects, cfDNA hyper-/hypo-methylation analysis by methods and systems provided herein may be used for diagnosis of, or detection of, or measuring for other types of diseases such as multiple sclerosis, traumatic/ischemic brain damage, diabetes, pancreatitis, or Alzheimer's disease, or infectious diseases (viral, bacterial, fungal, and so forth).

In some aspects, cfDNA hyper-/hypo-methylation analysis by methods and systems provided herein may be used to inform the microbiome composition, such as bacteria, fungi, viruses, and/or protozoa, in the subject, which may be used to inform the risk of infectious diseases or other health conditions.

It is contemplated that any aspect discussed in this specification can be implemented with respect to any method, system, kit, computer-readable medium, or apparatus of the present disclosure, and vice versa. Furthermore, apparatuses used in the disclosure can be used to achieve methods of the disclosure.

In some aspects, the method further comprises producing a report, such as electronically outputting a report indicative of hyper-/hypo-methylation profile. In some embodiments, the method further comprises processing the hyper-/hypo-methylation profile to generate a likelihood or risk of a subject as having or being suspected of having at least one disease or disorder. In some aspects, the disease or disorder is selected from the group consisting of cancer, multiple sclerosis, traumatic or ischemic brain damage, diabetes, pancreatitis, Alzheimer's disease, and fetal abnormality. In some aspects, the disease or disorder is a cancer selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, thyroid cancer, spleen cancer, gall bladder cancer, and prostate cancer.

In some aspects, one or more computer processors are individually or collectively programmed to electronically output a report indicative of hyper-/hypo-methylation profile. In some aspects, one or more computer processors are individually or collectively programmed to process the hyper-/hypo-methylation profile to generate a likelihood or risk of a subject as having or being suspected of having one or more diseases or disorders. In some aspects, the disease or disorder is selected from the group consisting of cancer, multiple sclerosis, traumatic or ischemic brain damage, diabetes, pancreatitis, Alzheimer's disease, and fetal abnormality. In some aspects, said disease or disorder is a cancer selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, thyroid cancer, spleen cancer, gall bladder cancer, and prostate cancer.

In another aspect, the present disclosure provides a non-transitory computer-readable medium comprising machine executable code that, upon execution by one or more computer processors, implements any of the methods disclosed herein. For example, the present disclosure provides a non-transitory computer-readable medium comprising machine executable code that, upon execution by one or more computer processors, implements a method for processing or analyzing a plurality of cfDNA molecules subjected to hyper-/hypo-methylation analysis provided by the present disclosure.

### V. Trained Algorithms

After identifying the counts of hyper-/hypo-methylated nucleic acid molecules in the targeted regions from nucleotide sequence information of a bodily sample (e.g., a cell-free biological sample) from one or more subjects, a trained algorithm may be used to process a test dataset (e.g., counts or normalized counts of hyper-/hypo-methylated nucleic acid molecules in the targeted regions of a test sample obtained or derived from a subject) to assess a disease or disorder state (e.g., detect a presence or absence of a disease or disorder) of the test subject. The trained algorithm may be configured to identify the disease or disorder state with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise an unsupervised machine learning algorithm.

The trained algorithm may be configured to accept a plurality of input variables and to produce one or more output values based on the plurality of input variables. The plurality of input variables may comprise one or more datasets indicative of a control or a disease or disorder state. For example, an input variable may comprise counts or normalized counts of hyper-/hypo-methylated nucleic acid molecules in the targeted regions corresponding to a disease or disorder state (e.g., having differential abundance for diseased samples vs. non-diseased samples). The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the cell-free biological sample by the classifier. The trained algorithm may comprise a binary classifier, such that each of the one or more output values comprises one of two values (e.g., {0, 1}, {positive, negative}, or {high-risk, low-risk}) indicating a classification of the cell-free biological sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the cell-free biological sample by the classifier.

The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's disease or disorder state, and may comprise, for example, a therapeutic intervention, a duration of the therapeutic intervention, and/or a dosage of the therapeutic intervention suitable to treat a disease or disorder. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof. For example, such descriptive labels may provide a prognosis of the disease or disorder state of the subject. As another example, such descriptive labels may provide a relative assessment of the disease or disorder state of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the disease or disorder state of the subject. Some numerical values may be mapped to descriptive labels, for example, by mapping 1 to “positive” and 0 to “negative.” Some of the output values may be assigned based on one or more cutoff values. For example, a binary classification of samples may assign an output value of “positive” or 1 if the sample indicates that the subject has at least a 50% probability of having a disease or disorder state (e.g., cancer). For example, a binary classification of samples may assign an output value of “negative” or 0 if the sample indicates that the subject has less than a 50% probability of having a disease or disorder state (e.g., cancer). In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject has a probability of having a disease or disorder state (e.g., cancer) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having a disease or disorder state (e.g., cancer) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a disease or disorder state (e.g., cancer) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a disease or disorder state (e.g., cancer) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a cell-free biological sample from a subject, associated datasets obtained by assaying the cell-free biological sample (as described elsewhere herein), and one or more known output values corresponding to the cell-free biological sample (e.g., a clinical diagnosis, prognosis, absence, or treatment efficacy of a disease or disorder state of the subject). Independent training samples may comprise cell-free biological samples and associated datasets and outputs obtained or derived from a plurality of different subjects. Independent training samples may comprise cell-free biological samples and associated datasets and outputs obtained at a plurality of different time points from the same subject (e.g., on a regular basis such as weekly, biweekly, or monthly). Independent training samples may be associated with the presence of the disease or disorder state (e.g., training samples comprising cell-free biological samples and associated datasets and outputs obtained or derived from a plurality of subjects known to have the disease or disorder state). Independent training samples may be associated with the absence of the disease or disorder state (e.g., training samples comprising cell-free biological samples and associated datasets and outputs obtained or derived from a plurality of subjects who are known to not have a previous diagnosis of the disease or disorder state or who have received a negative test result for the disease or disorder state).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise cell-free biological samples associated with the presence of the disease or disorder state and/or cell-free biological samples associated with the absence of the disease or disorder state. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with the presence of the disease or disorder state. In some aspects, the cell-free biological sample is independent of samples used to train the trained algorithm.

The trained algorithm may be trained with a first number of independent training samples associated with the presence of the disease or disorder state and a second number of independent training samples associated with the absence of the disease or disorder state. The first number of independent training samples associated with the presence of the disease or disorder state may be no more than the second number of independent training samples associated with the absence of the disease or disorder state. The first number of independent training samples associated with the presence of the disease or disorder state may be equal to the second number of independent training samples associated with the absence of the disease or disorder state. The first number of independent training samples associated with the presence of the disease or disorder state may be greater than the second number of independent training samples associated with the absence of the disease or disorder state.

The trained algorithm may be configured to identify the disease or disorder state at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the disease or disorder state by the trained algorithm may be calculated as the percentage of independent test samples (e.g., subjects known to have the disease or disorder state or subjects with negative clinical test results for the disease or disorder state) that are correctly identified or classified as having or not having the disease or disorder state.

The trained algorithm may be configured to identify the disease or disorder state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the disease or disorder state using the trained algorithm may be calculated as the percentage of cell-free biological samples identified or classified as having the disease or disorder state corresponding to subjects that truly have the disease or disorder state.

The trained algorithm may be configured to identify the disease or disorder state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the disease or disorder state using the trained algorithm may be calculated as the percentage of cell-free biological samples identified or classified as not having the disease or disorder state corresponding to subjects that truly do not have the disease or disorder state.

The trained algorithm may be configured to identify the disease or disorder state with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the disease or disorder state using the trained algorithm may be calculated as the percentage of independent test samples associated with the presence of the disease or disorder state (e.g., subjects known to have the disease or disorder state) that are correctly identified or classified as having the disease or disorder state.

The trained algorithm may be configured to identify the disease or disorder state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the disease or disorder state using the trained algorithm may be calculated as the percentage of independent test samples associated with the absence of the disease or disorder state (e.g., subjects with negative clinical test results for the disease or disorder state) that are correctly identified or classified as not having the disease or disorder state.

The trained algorithm may be configured to identify the disease or disorder state with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying cell-free biological samples as having or not having the disease or disorder state.

The trained algorithm may be adjusted or tuned to improve one or more of the performance, accuracy, PPV, NPV, clinical sensitivity, clinical specificity, or AUC of identifying the disease or disorder state. The trained algorithm may be adjusted or tuned by adjusting parameters of the trained algorithm (e.g., a set of cutoff values used to classify a cell-free biological sample as described elsewhere herein, or weights of a neural network). The trained algorithm may be adjusted or tuned continuously during the training process or after the training process has completed.

After the trained algorithm is initially trained, a subset of the inputs may be identified as the most influential or most important to be included for making high-quality classifications. For example, a subset of the counts or normalized counts of hyper-/hypo-methylated nucleic acid molecules in the targeted regions may be identified as most influential or most important to be included for making high-quality classifications or identifications of disease or disorder states (or sub-types of disease or disorder states). The set of counts or normalized counts of hyper-/hypo-methylated nucleic acid molecules in the targeted regions or a subset thereof may be ranked based on classification metrics indicative of each count's influence or importance toward making high-quality classifications or identifications of disease or disorder states (or sub-types of disease or disorder states). Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables (e.g., counts or normalized counts of hyper-/hypo-methylated nucleic acid molecules in the targeted regions) in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables (e.g., counts or normalized counts of hyper-/hypo-methylated nucleic acid molecules in the targeted regions) and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

After using a trained algorithm to process the dataset, the disease or disorder state (e.g., cancer) may be identified or monitored in the subject. The identification may be based at least in part on counts or normalized counts of hyper-/hypo-methylated nucleic acid molecules in the targeted regions having differential power for a given disease or disorder.

The disease or disorder state may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the disease or disorder state by the trained algorithm may be calculated as the percentage of independent test samples (e.g., subjects known to have the disease or disorder state or subjects with negative clinical test results for the disease or disorder state) that are correctly identified or classified as having or not having the disease or disorder state.

The disease or disorder state may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the disease or disorder state using the trained algorithm may be calculated as the percentage of cell-free biological samples identified or classified as having the disease or disorder state that correspond to subjects that truly have the disease or disorder state.

The disease or disorder state may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the disease or disorder state using the trained algorithm may be calculated as the percentage of cell-free biological samples identified or classified as not having the disease or disorder state that correspond to subjects that truly do not have the disease or disorder state.

The disease or disorder state may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the disease or disorder state using the trained algorithm may be calculated as the percentage of independent test samples associated with the presence of the disease or disorder state (e.g., subjects known to have the disease or disorder state) that are correctly identified or classified as having the disease or disorder state.

The disease or disorder state may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the disease or disorder state using the trained algorithm may be calculated as the percentage of independent test samples associated with the absence of the disease or disorder state (e.g., subjects with negative clinical test results for the disease or disorder state) that are correctly identified or classified as not having the disease or disorder state.

After the disease or disorder state is identified in a subject, a sub-type of the disease or disorder state (e.g., selected from among a plurality of sub-types of the disease or disorder state) may further be identified. The sub-type of the disease or disorder state may be determined based at least in part on counts or normalized counts of hyper-/hypo-methylated nucleic acid molecules in the targeted regions having differential power for a given disease or disorder. For example, the subject may be identified as being at elevated risk of a sub-type of cancer (e.g., selected from among a plurality of sub-types of a given cancer). After identifying the subject as being at elevated risk of a sub-type of disease, a clinical intervention for the subject may be selected based at least in part on the sub-type of disease for which the subject is identified as being at elevated risk. In some aspects, the clinical intervention is selected from a plurality of clinical interventions (e.g., clinically indicated for different sub-types of cancer). For example, the clinical intervention may be chemotherapy, radiotherapy, targeted therapy, or immunotherapy that is clinically indicated for the identified sub-type of a given cancer, but that is not clinically indicated for other sub-types of the given cancer.

In some aspects, the trained algorithm may determine that the subject is at elevated risk of the disease or disorder of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm may determine that the subject is at elevated risk of the disease or disorder at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

Upon identifying the subject as having the disease or disorder state, the subject may be optionally provided with a therapeutic intervention (e.g., prescribing an appropriate course of treatment to treat the disease or disorder state of the subject). The therapeutic intervention may comprise the administering of an effective dose of a drug, further testing or evaluation of the disease or disorder state, further monitoring of the disease or disorder state, an induction or inhibition of labor, or a combination thereof. If the subject is currently being treated for the disease or disorder state with a course of treatment, the therapeutic intervention may comprise a subsequent different course of treatment (e.g., to increase treatment efficacy due to the non-efficacy of the current course of treatment).

The therapeutic intervention may comprise recommending the subject for a secondary clinical test to confirm a diagnosis of the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

The counts or normalized counts of hyper-/hypo-methylated nucleic acid molecules in the targeted regions having differential power for a given disease or disorder may be assessed over a duration of time to monitor a patient (e.g., a subject who has a disease or disorder state or who is being treated for a disease or disorder state). In such cases, the counts or normalized counts of hyper-/hypo-methylated nucleic acid molecules in the targeted regions of the dataset of the patient may change during the course of treatment. For example, the counts or normalized counts of hyper-/hypo-methylated nucleic acid molecules in the targeted regions of the dataset of a patient with decreasing risk of the disease or disorder state due to effective treatment may shift toward the profile or distribution of a healthy subject (e.g., a subject without disease or disorder). Conversely, for example, the quantitative measures of the dataset of a patient with an increasing risk of the disease or disorder state due to an ineffective treatment may shift toward the profile or distribution of a subject with a higher risk of the disease or disorder state or a more advanced disease or disorder state.

The disease or disorder state of the subject may be monitored by monitoring a course of treatment for treating the disease or disorder state of the subject. The monitoring may comprise assessing the disease or disorder state of the subject at two or more time points. The assessment may be based at least on the counts or normalized counts of hyper-/hypo-methylated nucleic acid molecules in the targeted regions determined at each of the two or more time points.

In some aspects, a difference in the counts or normalized counts of hyper-/hypo-methylated nucleic acid molecules in the targeted regions determined between the two or more time points may be indicative of one or more clinical indications, such as (i) a diagnosis of the disease or disorder state of the subject, (ii) a prognosis of the disease or disorder state of the subject, (iii) an increased risk of the disease or disorder state of the subject, (iv) a decreased risk of the disease or disorder state of the subject, (v) an efficacy of the course of treatment for treating the disease or disorder state of the subject, and (vi) a non-efficacy of the course of treatment for treating the disease or disorder state of the subject.

In some aspects, a difference in the counts or processed counts of hyper-/hypo-methylated nucleic acid molecules in the targeted regions determined between the two or more time points may be indicative of a diagnosis of the disease or disorder state of the subject. For example, if the disease or disorder state was not detected in the subject at an earlier time point but was detected in the subject at a later time point, then the difference is indicative of a diagnosis of the disease or disorder state of the subject. A clinical action or decision may be made based on this indication of diagnosis of the disease or disorder state of the subject, such as, for example, prescribing a new therapeutic intervention for the subject. The clinical action or decision may comprise recommending the subject for a secondary clinical test to confirm the diagnosis of the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

In some aspects, a difference in the counts or normalized counts of hyper-/hypo-methylated nucleic acid molecules in the targeted regions determined between the two or more time points may be indicative of a prognosis of the disease or disorder state of the subject.

In some aspects, a difference in the counts or normalized counts of hyper-/hypo-methylated nucleic acid molecules in the targeted regions between the two or more time points may be indicative of the subject having an increased risk of the disease or disorder state. For example, if the disease or disorder state was detected in the subject both at an earlier time point and at a later time point, and if the difference is a positive difference (e.g., counts or normalized counts of hyper-/hypo-methylated nucleic acid molecules in the targeted regions increased from the earlier time point to the later time point), then the difference may be indicative of the subject having an increased risk of the disease or disorder state. A clinical action or decision may be made based on this indication of the increased risk of the disease or disorder state, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

In some aspects, a difference in the counts or normalized counts of hyper-/hypo-methylated nucleic acid molecules in the targeted regions determined between the two or more time points may be indicative of the subject having a decreased risk of the disease or disorder state. For example, if the disease or disorder state was detected in the subject both at an earlier time point and at a later time point, and if the difference is a negative difference (e.g., the counts or normalized counts of hyper-/hypo-methylated nucleic acid molecules in the targeted regions decreased from the earlier time point to the later time point), then the difference may be indicative of the subject having a decreased risk of the disease or disorder state. A clinical action or decision may be made based on this indication of the decreased risk of the disease or disorder state (e.g., continuing or ending a current therapeutic intervention) for the subject. The clinical action or decision may comprise recommending the subject for a secondary clinical test to confirm the decreased risk of the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

In some aspects, a difference in the counts or normalized counts of hyper-/hypo-methylated nucleic acid molecules in the targeted regions determined between the two or more time points may be indicative of an efficacy of the course of treatment for treating the disease or disorder state of the subject. For example, if the disease or disorder state was detected in the subject at an earlier time point but was not detected in the subject at a later time point, then the difference may be indicative of an efficacy of the course of treatment for treating the disease or disorder state of the subject. A clinical action or decision may be made based on this indication of the efficacy of the course of treatment for treating the disease or disorder state of the subject, e.g., continuing or ending a current therapeutic intervention for the subject. The clinical action or decision may comprise recommending the subject for a secondary clinical test to confirm the efficacy of the course of treatment for treating the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

In some aspects, a difference in the counts or normalized counts of hyper-/hypo-methylated nucleic acid molecules in the targeted regions determined between the two or more time points may be indicative of a non-efficacy of the course of treatment for treating the disease or disorder state of the subject. For example, if the disease or disorder state was detected in the subject both at an earlier time point and at a later time point, and if the difference is a positive or zero difference (e.g., the counts or normalized counts of hyper-/hypo-methylated nucleic acid molecules in the targeted regions increased or remained at a constant level from the earlier time point to the later time point), and if an efficacious treatment was indicated at an earlier time point, then the difference may be indicative of a non-efficacy of the course of treatment for treating the disease or disorder state of the subject. A clinical action or decision may be made based on this indication of the non-efficacy of the course of treatment for treating the disease or disorder state of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

In some aspects, for example, the clinical health data comprises one or more quantitative measures of the subject, such as age, weight, height, body mass index (BMI), blood pressure, heart rate, glucose levels, previous history or family history of disease (e.g., cancer). As another example, the clinical health data can comprise one or more categorical measures, such as race, ethnicity, history of medication or other clinical treatment, history of tobacco use, history of alcohol consumption, daily activity or fitness level, genetic test results, blood test results, and imaging results.

In some aspects, the methods provided herein are performed using a computer or mobile device application. For example, a subject can use a computer or mobile device application to input her own clinical health data, including quantitative and/or categorical measures. The computer or mobile device application can then use a trained algorithm to process the clinical health data. The computer or mobile device application can then display a report indicative of the results of the computer-implemented method.

In some aspects, the detected disease or disorder state of the subject can be refined by performing one or more subsequent clinical tests for the subject. For example, the subject can be referred by a physician for one or more subsequent clinical tests based on the initial detected disease or disorder state. This subsequent clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

After the disease or disorder state is identified or monitored in the subject, a report may be electronically output that is indicative of (e.g., identifies or provides an indication of) the disease or disorder state of the subject. The subject may not display a disease or disorder state (e.g., is asymptomatic of the disease or disorder state). The report may be presented on a graphical user interface (GUI) of an electronic device of a user. The user may be the subject, a caretaker, a physician, a nurse, or another health care worker.

The report may include one or more clinical indications such as (i) a diagnosis of the disease or disorder state of the subject, (ii) a prognosis of the disease or disorder state of the subject, (iii) an increased risk of the disease or disorder state of the subject, (iv) a decreased risk of the disease or disorder state of the subject, (v) the efficacy of the course of treatment for treating the disease or disorder state of the subject, and (vi) the non-efficacy of the course of treatment for treating the disease or disorder state of the subject. The report may include one or more clinical actions or decisions made based on these one or more clinical indications. Such clinical actions or decisions may be directed to therapeutic interventions, or further clinical assessment or testing of the disease or disorder state of the subject.

For example, a clinical indication of a diagnosis of the disease or disorder state of the subject may be accompanied with a clinical action of prescribing a new therapeutic intervention for the subject. As another example, a clinical indication of an increased risk of the disease or disorder state of the subject may be accompanied with a clinical action of prescribing a new therapeutic intervention or switching therapeutic interventions (e.g., ending a current treatment and prescribing a new treatment) for the subject. As another example, a clinical indication of a decreased risk of the disease or disorder state of the subject may be accompanied with a clinical action of continuing or ending a current therapeutic intervention for the subject. As another example, a clinical indication of the efficacy of the course of treatment for treating the disease or disorder state of the subject may be accompanied with a clinical action of continuing or ending a current therapeutic intervention for the subject. As another example, a clinical indication of a non-efficacy of the course of treatment for treating the disease or disorder state of the subject may be accompanied with a clinical action of ending a current therapeutic intervention and/or switching to (e.g., prescribing) a different new therapeutic intervention for the subject.

### VI. Kits of the Disclosure

The present disclosure provides a kit comprising any of the compositions described herein. In a non-limiting example, one or more of the following may be included in a kit: substrates for capturing nucleic acids (which may be referred to as panels or arrays); cfDNA; one or more apparatuses for collection of cfDNA; targeted probes; enzymes; adapters; primers (e.g., PCR primers); deoxynucleoside triphosphates (dNTPs); hybridization buffer; wash buffers; 20× saline-sodium citrate (SSC) buffer; other chemicals and compositions, including adenosine triphosphate (ATP), dithiothreitol (DTT), and so forth; and any combination thereof.

The components of the kits may be packaged either in aqueous media or in lyophilized form. The kit may comprise a container, such as at least one vial, test tube, flask, bottle, or another container, into which a component may be placed and/or suitably aliquoted. Where there is more than one component in the kit, the kit may comprise a second, third or other additional container into which the additional components may be separately placed. However, various combinations of components may be comprised in a vial. The kits of the present disclosure may comprise a container for containing component(s) in close confinement for commercial sale. Such containers may include blow-molded plastic containers into which the desired vials are retained.

Kits of the present disclosure may include instructions for performing methods provided herein, such as methods for hybridizing the cfDNA to probes and preparing a sequencing library for hyper-/hypo-methylation analysis. Such instructions may be in physical form (e.g., printed instructions) or electronic form.

Kits of the present disclosure may include a software package or a web link to a server or cloud-computing platform for analyzing the data generated with the kit. The analysis may provide information about the quality control of the kits such as hybridization efficiency, and provide hyper-/hypo-methylation counts profile of the cfDNA in the targeted regions.

Kits of the present disclosure may include a report generated by a software package provided with the kit, or by a server or cloud-computing platform. The report may provide information for (1) diagnosis and/or prophylaxis of a medical condition; (2) therapy for a medical condition; (3) therapy monitoring; and so forth. For example, the report may provide information about the presence or risk of cancer, including of a particular type of cancer.

### VII. Computer Systems

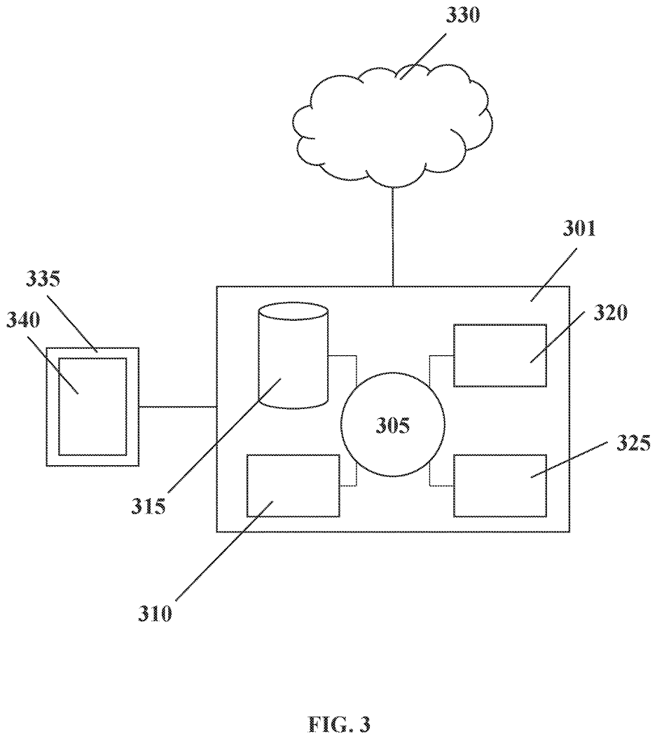

The present disclosure provides computer systems that are programmed to implement methods of disclosure. FIG. 3 shows a computer system 301 that is programmed or otherwise configured to, for example, process sequencing or imaging data to identify the counts or normalized counts of hyper-/hypo-methylated nucleic acid molecules in each of the targeted regions, input counts in these targeted regions as features for one or more trained classifiers, generate a likelihood of a subject as having or being suspected of having a disease or disorder, analyze nucleotide sequence information, train classifiers using a training data set and a set of counts or normalized counts of hyper-/hypo-methylated nucleic acid molecules in the targeted regions, obtain or generate sequencing data of cfDNA samples, perform a clustering method to identify a set of counts, and determine the accuracy of trained classifiers in assessing disease status. The computer system 301 can regulate various aspects of analysis, calculation, and generation of the present disclosure, such as, for example, processing sequencing to identify the counts or normalized counts of hyper-/hypo-methylated nucleic acid molecules in each targeted region, inputting counts as features for one or more trained classifiers, generating a likelihood of a subject as having or being suspected of having a disease or disorder, analyzing nucleotide sequence information, training classifiers using a training data set and a set of counts or normalized counts of hyper-/hypo-methylated nucleic acid molecules in the targeted regions, obtaining or generating sequencing data of cfDNA samples, performing a clustering method to identify a set of counts, and determining the accuracy of trained classifiers in assessing disease status. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single-core or multi-core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an intranet and/or extranet, or an intranet and/or extranet that is in communication with the Internet. The network 330 in some cases is a telecommunication and/or data network. The network 330 can include one or more computer servers, which can enable distributed computing, such as cloud computing. For example, one or more computer servers may enable cloud computing over the network 330 (“the cloud”) to perform various aspects of analysis, calculation, and generation of the present disclosure, such as, for example, processing sequencing or imaging data to identify the counts or normalized counts of hyper-/hypo-methylated nucleic acid molecules in each targeted regions, inputting counts as features for one or more trained classifiers, generating a likelihood of a subject as having or being suspected of having a disease or disorder, analyzing nucleotide sequence information, training classifiers using a training data set and a set of counts or normalized counts of hyper-/hypo-methylated nucleic acid molecules in the targeted regions, obtaining or generating sequencing data of cfDNA samples, performing a clustering method to identify a set of counts or normalized counts of hyper-/hypo-methylated nucleic acid molecules in the targeted regions, and determining the accuracy of trained classifiers in assessing disease status. Such cloud computing may be provided by cloud computing platforms such as, for example, Amazon Web Services (AWS), Microsoft Azure, Google Cloud Platform, and IBM cloud. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application-specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user (e.g., a physician, a nurse, a caretaker, a patient, or a subject). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine-executable or machine-readable code can be provided in the form of software. During use, the code can be executed by the processor 305. In some cases, the code can be retrieved from the storage unit 315 and stored in the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer- or machine- “readable medium” refer to any medium that participates in providing instructions to a processor for execution.

Hence, a machine-readable medium, such as computer-executable code, may take many forms, including but not limited to, a tangible storage medium, a carrier wave medium, or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as the main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 301 can include or be in communication with an electronic display 835 that comprises a user interface (UI) 340 for providing, for example, the hyper-/hypo-methylation counts profile, a report indicative of the counts profile, and/or a likelihood of a subject as having or being suspected of having a disease or disorder. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, process sequencing or imaging data to identify the counts or normalized counts of hyper-/hypo-methylated nucleic acid molecules in each of the targeted regions, input counts as features for one or more trained classifiers, generate a likelihood of a subject as having or being suspected of having a disease or disorder, analyze nucleotide sequence information, train classifiers using a training data set and a set of counts or normalized counts of hyper-/hypo-methylated nucleic acid molecules in the targeted regions, obtain or generate sequencing data of cfDNA samples, perform a clustering method to identify a set of counts or normalized counts of hyper-/hypo-methylated nucleic acid molecules in the targeted regions, and determine the accuracy of trained classifiers in assessing disease status.

While preferred aspects of the present invention have been shown and described herein, it will be obvious to those skilled in the art that such aspects are provided by way of example only. It is not intended that the invention be limited by the specific examples provided within the specification. While the invention has been described with reference to the aforementioned specification, the descriptions and illustrations of the aspects herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the aspects of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

Although the present disclosure and its advantages have been described in detail, it should be understood that various changes, substitutions, and alterations can be made herein without departing from the spirit and scope of the design as defined by the appended claims. Moreover, the scope of the present application is not intended to be limited to the particular aspects of the process, machine, manufacture, composition of matter, means, methods, and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding aspects described herein may be utilized according to the present disclosure. Accordingly, the appended claims are intended to include within their scopes such as processes, machines, manufacture, compositions of matter, means, methods, or steps.

### VIII. Certain Assay Methods

### A. Detection of Methylated DNA

Aspects of the methods include assaying nucleic acids to determine expression levels and/or methylation levels of nucleic acids. Aspects of the disclosure include the detection of one or more CpG islands, such as at least, at most, or exactly 1, 2, 3, 4, 5, 6, 7, 8, 9, or 10 CpG islands (or any range derivable therein). Each biomarker may comprise or consist of at least or at most or exactly 1, 2, 3, 4, 5, 6, 7, 8, 9, or 10 CpG islands (or any range derivable therein). Various assays may be used for the detection of methylated DNA. Exemplary methods are described herein.

### 1. Bisulfite Sequencing

The bisulfite treatment of DNA mediates the deamination of cytosine into uracil, and these converted residues may be read as thymine, as determined by PCR-amplification and subsequent Sanger sequencing analysis. However, 5 mC residues are resistant to this conversion and, so, may remain read as cytosine. Thus, comparing the Sanger sequencing read from an untreated DNA sample to the same sample following bisulfite treatment enables the detection of the methylated cytosines. With next-generation sequencing (NGS) technology, this approach can be extended to DNA methylation analysis across an entire genome. To ensure complete conversion of non-methylated cytosines, controls may be incorporated for bisulfite reactions.

Whole genome bisulfite sequencing (WGBS) is similar to whole genome sequencing, except for the additional step of bisulfite conversion. Sequencing of the 5 mC-enriched fraction of the genome is not only a less expensive approach, but it also allows one to increase the sequencing coverage and, therefore, precision in revealing differentially-methylated regions. Sequencing may be done using any existing NGS platform; Illumina and Life Technologies both offer kits for such analysis.

Bisulfite sequencing methods include reduced representation bisulfite sequencing (RRBS), where only a fraction of the genome is sequenced. In RRBS, enrichment of CpG-rich regions is achieved by isolation of short fragments after MspI digestion that recognizes CCGG sites (and it cut both methylated and unmethylated sites). It ensures isolation of ~85% of CpG islands in the human genome. Then, the same bisulfite conversion and library preparation is performed as for WGBS. The RRBS procedure normally requires ~100 ng-1 μg of DNA.

### 2. Hybridization

Methods of the present disclosure may use nucleic acids that hybridize to other nucleic acids under particular hybridization conditions. Various methods may be used for hybridizing nucleic acids. See, e.g., Current Protocols in Molecular Biology, John Wiley and Sons, N.Y. (1989), 6.3.1-6.3.6, which is incorporated by reference herein in its entirety. Methods of the present disclosure may use a moderately stringent hybridization condition using a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other may remain hybridized to each other.

The parameters affecting the choice of hybridization conditions and guidance for devising suitable conditions may be described by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), each of which is herein incorporated by reference in their entirety) and can be readily determined based on, for example, the length and/or base composition of the DNA.

### 3. Probes

In another aspect, nucleic acid molecules are suitable for use as primers or hybridization probes for the detection or purification of nucleic acid sequences.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to isolate or purify certain nucleic acids.

### B. Sequencing

DNA, including bisulfite-converted DNA, may be used for the amplification of the region of interest followed by sequencing. Primers can be designed around the CpG island and used for PCR amplification of bisulfite-converted DNA. The resulting PCR products may be cloned and sequenced. Accordingly, aspects of the disclosure may include sequencing nucleic acids to detect methylation of nucleic acids and/or biomarkers. In some aspects, the methods of the disclosure include a sequencing method. Sequencing methods useful for certain aspects may include those described below. Other sequencing methods may also be used, in some aspects.

1. Massively Parallel Signature Sequencing (mpss).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these may be used for sequencing cDNA for measurements of gene expression levels. Indeed, the powerful Illumina HiSeq2000, HiSeq2500 and MiSeq systems are based on MPSS.

### 2. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLID platform, which is now owned by Life Technologies.

### 3. 454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLID on the other.

### 4. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massivelly parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

### 5. Solid Sequencing.

Applied Biosystems' (now a Thermo Fisher Scientific brand) SOLD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

### 6. Ion Torrent Semiconductor Sequencing.

Ion Torrent Systems Inc. (now owned by Thermo Fisher Scientific) developed a system based on using standard sequencing chemistry, but with a semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide, it is incorporated into the growing complementary strand. This causes the release of a hydrogen ion that triggers a hypersensitive ion sensor, which indicates that a reaction has occurred. If homopolymer repeats are present in the template sequence multiple nucleotides may be incorporated in a single cycle. This leads to a corresponding number of released hydrogens and a proportionally higher electronic signal.

### 7. Dna Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology may be used for multiple genome sequencing projects.

### 8. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

### 9. Single Molecule Real Time (SMRT) Sequencing.

SMRT sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.]

### C. Additional Assay Methods

In some aspects, methods involve amplifying and/or sequencing one or more target genomic regions using at least one pair of primers specific to the target genomic regions. In certain aspects, the primers are 7, 8, 9, 10, 11, 12, 13, 14, 15, 16, 17, 18, 19, 20, 21, 22, 23, 24, or more (or any range derivable therein) nucleotides. In other aspects, enzymes are added such as primases or primase/polymerase combination enzyme to the amplification step to synthesize primers.

In some aspects, arrays can be used to detect nucleic acids of the disclosure. An array comprises a solid support with nucleic acid probes attached to the support. Arrays may comprise a plurality of different nucleic acid probes that are coupled to a surface of a substrate in different, known locations. These arrays, also described as “microarrays” or colloquially “chips”, may be described by, for example, U.S. Pat. Nos. 5,143,854, 5,445,934, 5,744,305, 5,677,195, 6,040,193, 5,424,186, and Fodor et al., 1991), each of which is incorporated by reference in its entirety. Techniques for the synthesis of these arrays using mechanical synthesis methods are described in, e.g., U.S. Pat. No. 5,384,261, incorporated herein by reference in its entirety. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is incorporated by reference herein in their entirety.

In addition to the use of arrays and microarrays, it is contemplated that a number of difference assays may be employed to analyze nucleic acids. Such assays include, but are not limited to, nucleic amplification, polymerase chain reaction, quantitative PCR, RT-PCR, in situ hybridization, digital PCR, dd PCR (digital droplet PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a genomic region, biomarker, probe, or oligo described herein. The term “primer” or “probe” as used herein, is meant to encompass any nucleic acid that is capable of priming the synthesis of a nascent nucleic acid in a template-dependent process and/or pairing with a single strand of an oligo of the disclosure, or portion thereof. Primers may be oligonucleotides from ten to twenty and/or thirty nucleic acids in length, but longer sequences can be employed. Primers may be provided in double-stranded and/or single-stranded form.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical approaches or by introducing selected sequences into recombinant vectors for recombinant production.

In one aspect, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

For applications requiring high selectivity, one may desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and may be particularly suitable for isolating specific genes or for detecting specific mRNA transcripts. It is generally appreciated that conditions can be rendered more stringent by the addition of increasing amounts of formamide.

In one aspect, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one aspect, the PCR amplification utilizes one or more internal PCR standards. The internal standard may be an abundant housekeeping gene in the cell or it can specifically be GAPDH, GUSB and B-2 microglobulin. These standards may be used to normalize expression levels so that the expression levels of different gene products can be compared directly. An internal standard may be used to normalize expression levels.

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results may be achieved. All such similar substitutes and modifications apparent to those skilled in the art are deemed to be within the spirit, scope and concept of the invention as defined by the appended claims.

What is claimed is:

1. A method for enriching hyper-methylated and/or hypo-methylated nucleic acid molecules in a plurality of nucleic acid molecules, comprising:
(a) ligating a first set of adapters to each end of each nucleic acid in the plurality of nucleic acid molecules to generate a first set of ligated molecules;
(b) digesting the first set of ligated molecules with one or more methylation-sensitive restriction enzymes and/or methylation-dependent restriction enzymes to produce a plurality of nucleic acid molecules with digestion-enzyme-specific overhangs, and ligating a second set of adapters to said plurality of nucleic acid molecules with digestion-enzyme-specific overhangs to generate a second set of ligated molecules, wherein the second set of ligated molecules do not have a sequence that is recognized by the one or more restriction enzymes;
(c) amplifying the first set of ligated molecules and the second set of ligated molecules to produce amplified adapter-ligated DNA fragments, at least in part using one or more primers that bind to the first set of adapters and one or more primers that bind to the second set of adapters;
(d) partitioning the amplified adapter-ligated DNA fragments into at least two partitions; and
(e) enriching the amplified adapter-ligated DNA fragments that have the first set of adapters on both ends in a first partition to generate enriched DNA fragments in the first partition.

2. The method of claim 1, wherein the plurality of nucleic acid molecules comprises cell-free DNA.

3. The method of claim 1 or 2, further comprising enriching hyper-methylated nucleic acid molecules, wherein (b) further comprises digesting the plurality of nucleic acid molecules with one or more methylation-sensitive restriction enzymes to produce a plurality of nucleic acid molecules with digestion-enzyme-specific overhangs, and ligating the second set of adapters to said plurality of nucleic acid molecules with digestion-enzyme-specific overhangs to generate a second set of ligated molecules, wherein the second set of ligated molecules do not have a sequence that is recognized by the one or more methylation-sensitive restriction enzymes.

4. The method of claim 1 or 2, further comprising enriching hypo-methylated nucleic acid molecules, wherein (b) further comprises digesting the plurality of nucleic acid molecules with one or more methylation-dependent restriction enzymes to produce a plurality of nucleic acid molecules with digestion-enzyme-specific overhangs, and ligating the second set of adapters to said plurality of nucleic acid molecules with digestion-enzyme-specific overhangs to generate a second set of ligated molecules, wherein the second set of ligated molecules do not have a sequence that is recognized by the one or more methylation-dependent restriction enzymes.

5. The method of any one of claims 1-4, wherein the one or more methylation-sensitive restriction enzymes comprise one or more of HhaI, HpyCH4IV, AclI, AcII, AfeI, AgeI, AccII, AatII, Aor13HI, Aor51HI, AscI, AsiSI, AvaI, BceAI, BmgBI, BsaAI, BsaHI, BsiEI, BsiWI, BsmBI, BspDI, BspEI, BspT104I, BsrBI, BssHII, BstUI, Cfr10I, ClaI, CpoI, Eco52I, HaeII, HgaI, HinP1I, HpaII, Hpy99I, KasI, KroNI, MluI, Nael, NarI, NgoMIV, NotI, NruI, NsbI, PaeR7I, PmaCI, PmlI, Psp1406I, Pvul, RsrII, SacII, SalI, SamI, SnaBI, or a functional analog thereof, or a mixture thereof.

6. The method of any one of claims 1-5, wherein the one or more methylation-dependent restriction enzymes comprise one or more of LpnPI, McrBC, GlaI, PkrI, MteI, AoxI, or a functional analog thereof, or a mixture thereof.

7. The method of any one of claims 1-6, wherein the second set of adapters have overhangs that are complementary to overhangs generated by the methylation-sensitive restriction enzymes and/or methylation-dependent restriction enzymes, and wherein (b) further comprises digesting, with one or more additional restriction enzymes, adapters from the first set of adapters and/or second set of adapters that have ligated together, while not digesting a junction between a DNA fragment and an adapter.

8. The method of claim 7, wherein the one or more additional restriction enzymes comprise one or more of BspDI, ClaI, AclI, NarI, Xhol, SmlI, HpyF30I, PaeR7I, Sfr274I, or a functional analog thereof, or a mixture thereof.

9. The method of any one of claims 1-8, wherein digesting the plurality of nucleic acid molecules and ligating the second set of adapters occur in different reaction vessels.

10. The method of any one of claims 1-9, further comprising subjecting the plurality of nucleic acid molecules to conditions sufficient to permit methylated nucleic acid bases to be distinguishable from unmethylated nucleic acid bases before (c) and after (b).

11. The method of claim 10, wherein subjecting the plurality of nucleic acid molecules to conditions sufficient to permit methylated nucleic acid bases to be distinguishable from unmethylated nucleic acid bases comprises subjecting the plurality of nucleic acid molecules to one or more enzymatic or chemical reactions.

12. The method of claim 10 or 11, wherein subjecting the plurality of nucleic acid molecules to conditions sufficient to permit the methylated nucleic acid bases to be distinguishable from the unmethylated nucleic acid bases comprises subjecting the plurality of nucleic acid molecules to bisulfite conversion.

13. The method of any one of claims 1-12, wherein the amplifying in (c) comprises between 1 to 3, 1 to 5, or 1 to 10 amplification cycles.

14. The method of any one of claims 1-13, further comprising enriching the amplified adapter-ligated DNA fragments that have the second set of adapters on both ends in a second partition to generate enriched DNA fragments in the second partition.

15. The method of any one of claims 1-14, further comprising amplifying the first partition of amplified adapter-ligated DNA fragments using primers that are capable of initiating polymerization at the first set of adapters.

16. The method of claim 14 or 15, further comprising amplifying a second partition of amplified adapter-ligated DNA fragments using primers that are capable of initiating polymerization at the second set of adapters.

17. The method of any one of claims 1-16, wherein (d) further comprises partitioning the amplified adapter-ligated DNA fragments into three or more partitions, and wherein the method further comprises enriching the amplified adapter-ligated DNA fragments in a third partition that have the first set of adapters in one end and have the second set of adapters in another end to generate enriched DNA fragments in the third partition.

18. The method of any one of claims 1-17, further comprising amplifying, in a third partition, amplified adapter-ligated DNA fragments that have the first set of adapters in one end and have the second set of adapters in another end, using primers that are capable of initiating polymerization at the first set of adapters in one end and initiating polymerization at the second set of adapters in another end of the amplified adapter-ligated DNA fragments.

19. The method of any one of claims 1-18, further comprising capturing at least a subset of the amplified adapter-ligated DNA fragments in one or more targeted genomic regions using hybrid capture probes.

20. The method of claim 19, wherein the hybrid capture probes cover one or more restriction enzyme cutting sites in the targeted genomic regions.

21. The method of any one of claims 1-20, further comprising (f) processing the enriched DNA fragments to determine a methylation status of the enriched DNA fragments.

22. The method of claim 21, wherein (f) further comprises generating sequencing data that provides counts of the enriched DNA fragments.

23. The method of claim 21 or 22, wherein (b) is performed using one or more methylation-sensitive restriction enzymes, and wherein (f) further comprises determining the methylation status by counting hyper-methylated DNA fragments in a first partition and counting hypo-methylated DNA fragments in a second partition.

24. The method of any one of claims 21-23, wherein (b) is performed using one or more methylation-dependent restriction enzymes, and wherein (f) further comprises determining the methylation status by counting hypo-methylated DNA fragments in a first partition and counting hyper-methylated DNA fragments in a second partition.

25. The method of any one of claims 21-24, wherein the methylation status is indicative of the presence or absence of a disease or risk thereof in the individual.

26. The method of claim 25, wherein the presence or absence of a disease or disorder of the subject is detected using one or more trained machine-learning classifiers.

27. The method of claim 26, wherein the one or more trained machine-learning classifiers comprises features comprising counts of the enriched DNA fragments in (e).

28. The method of claim 26 or 27, wherein the one or more trained machine-learning classifiers comprise at least one of support vector machine, random forest, k-nearest neighbor, naïve Bayes, Gaussian process, decision trees, XGBoost, neural networks, linear and quadratic discrimination analysis, logistic regression, general linear models, and any combination thereof.

29. The method of any one of claims 25-28, further comprising administering a clinical intervention to the subject, based at least in part on the presence or absence of the disease or the risk thereof in the individual.

30. The method of claim 29, wherein the clinical intervention comprises an effective amount of a chemotherapy, a radiation therapy, a hormone therapy, a targeted therapy, an immunotherapy, a biologic, a cell therapy, an antibiotic, an anti-viral, radiation, cryoablation, or a combination thereof, and/or a surgical intervention.

31. The method of any one of claims 25-29, wherein the disease comprises cancer, an infectious disease, or a non-communicable disease.

32. A kit comprising reagents to perform the method of any one of claims 1-31.

33. A method of treating a subject, comprising:
(a) performing the method of any one of claims 1-31 on a biological sample from the subject to determine whether the subject has or does not have a condition sensitive to a clinical intervention; and
(b) administering the clinical intervention to the subject if the subject has been determined to have the condition sensitive to the clinical intervention.

34. The method of claim 33, wherein the subject has, or is suspected of having, cancer, an infectious disease, or a non-communicable disease.

35. The method of claim 33 or 34, wherein the condition sensitive to a clinical intervention is a neoplasm, a metastasis, an infection, or an autoimmune disorder.

36. The method of any one of claims 33-35, wherein the clinical intervention comprises an effective amount of a chemotherapy, a radiation therapy, a hormone therapy, a targeted therapy, an immunotherapy, a biologic, a cell therapy, an antibiotic, an anti-viral, radiation, cryoablation, or a combination thereof, and/or a surgical intervention.

37. The method of any one of claims 33-36, further comprising processing the enriched DNA fragments to determine the methylation status of the enriched DNA fragments, and wherein the methylation status is indicative of whether the patient has or does not have a condition sensitive to the clinical intervention.

38. A method of monitoring a response to a first clinical intervention in a subject, comprising:
(a) performing the method of any one of claims 1-31 on a biological sample from the subject, wherein the subject has received at least one round of the first clinical intervention;
(b) processing the enriched DNA fragments to determine the methylation status of the enriched DNA fragments; and
(c) administering an additional round of the first clinical intervention a second clinical intervention to the subject, based at least in part on the methylation status of the enriched DNA fragments.

39. The method of claim 38, wherein the subject has, or is suspected of having, cancer, an infectious disease, or a non-communicable disease.

40. The method of claim 38 or 39, wherein the clinical intervention comprises an effective amount of a chemotherapy, a radiation therapy, a hormone therapy, a targeted therapy, an immunotherapy, a biologic, a cell therapy, an antibiotic, an anti-viral, radiation, cryoablation, or a combination thereof, and/or a surgical intervention.

41. The method of any one of claims 38-40, wherein the a second clinical intervention is different from the first clinical intervention.

42. The method of any one of claim 38-41, wherein the second clinical intervention comprises an effective amount of a chemotherapy, a radiation therapy, a hormone therapy, a targeted therapy, an immunotherapy, a biologic, a cell therapy, an antibiotic, an anti-viral, radiation, cryoablation, or a combination thereof, and/or a surgical intervention, or halting of the first clinical intervention.

43. A method of diagnosing or prognosing a disease in a subject, the method comprising performing the method of any one of claims 1-31 to determine whether the subject has the disease or the severity of the disease in the subject.

44. The method of claim 43, wherein the disease is a cancer, an infectious disease, or a non-communicable disease.
